(19) United States
(12) Patent Application Publication
(10) Pub. No.: US 2005/0215907 A1
(43) Pub. Date: Sep. 29, 2005

(54) Ultrasonic transducer for electronic devices

(76) Inventors: Minoru Toda, Lawrenceville, NJ (US); Kyung-Tae Park, Berwyn, PA (US)

Correspondence Address: Plevy & Howard, 600 North Easton Road, Willow Grove, PA, 19090, US

(21) Appl. No.: 10/622,837

(22) Filed: Jul. 18, 2003

Related U.S. Application Data
(60) Provisional application No. 60/396,954, filed on Jul. 18, 2002.

(51) Int. Cl. A61B 8/14

(52) U.S. Cl. 600/459

(57) ABSTRACT

An ultrasound transducer for an electronic device, including a housing and an ultrasonic transducer element integrated with the housing. The transducer element is capable of operating in at least one of a receiver mode and transmitter mode. In the receiver mode, the transducer element produces an electrical signal in response to an impinging acoustic signal. In the transmitter mode, the transducer element produces an acoustic signal in response to an electrical signal applied thereto. The housing has at least one surface which ensures mechanical stressing of the transducer element in a manner which causes the transducer element to produce the signals.

### CLAIM FOR PRIORITY

This application claims the benefit under 35 USC 119(e) of U.S. Provisional Patent Application Ser. No. 60/396,954, filed Jul. 18, 2002, the entirety of which is hereby incorporated by reference.

### FIELD OF THE INVENTION

The invention relates generally to ultrasonic transducers and more particularly to ultrasonic transducers for use in electronic devices.

### BACKGROUND OF THE INVENTION

Communications between an ultrasonic transducer remotely mounted or positioned on a movable stylus, such as a moveable pen and other remotely located transducers (for example, transducers fixed at remote positions from the stylus) make it possible to determine the position of the pen and ultimately to reproduce information associated with stylus movement. Such relatively “fixed” equipment (in contrast to transducers mounted on a moving stylus) may nevertheless comprise portable electronic devices including, without limitation, cell phones, hand-held digital devices such as PDAs, notebook computers, games, or stand-alone equipment. Other devices may include keyboards for personal computers, telephones, and the like. The digital information associated with stylus position may be used, without limitation, for drawings, maps, or pictorial illustrations, as well as for e-mail, facsimile transmissions, document creations, document and file creation (in combination with a word processor), or input devices for computer games.

In each of these applications, it is desirable that the integration of the ultrasonic transducer in such electronic devices be accomplished such that the transducer is virtually invisible, and that the transducer be rugged and not susceptible to dust or dirt particles. Such features are particularly advantageous for portable electronic devices.

In addition, conventional small transducer assemblies typically provide low or undesirable sensitivities, are bulky, and manifest uncontrollable resonance frequency conditions.

Accordingly, an ultrasonic transducer that may be integrated into the housing of an electronic device such that the transducer is virtually invisible from an external vantage point and, which overcomes the aforementioned problems is highly desired.

It is also desirable to have such an ultrasonic transducer which is a modular component of an electronic device such that the transducer and its associated housing is insertable into a recessed region or receiving cavity of the electronic device as a modular unit, whereby, when inserted into the recessed region or receiving cavity, the transducer including its associated housing is flush with or recessed from the outer surface of the electronic device's housing. Still further, a transducer assembly that is thin, economical, easy to assemble, and has increased sensitivity is desired.

### BRIEF DESCRIPTION OF DRAWINGS

FIG. 1 is an embodiment of an embedded, ultrasonic transducer of the present invention.

FIGS. 2-5, 6A-E, 7A-B, 8A-B, 9A-B, 10A-B, 11, 12, and 13/14 are alternative embodiments of the embedded, ultrasonic transducer of the present invention.

FIG. 15 is an exemplary illustration of a digitizer system including ultrasonic transducers of the present invention.

FIG. 16 shows a cross-sectional view of an embodiment of a double-clamped ultrasonic transducer of the present invention.

FIG. 17 shows a front perspective view of the backplate of FIG. 16.

FIG. 18 shows a back perspective view of the front cover of FIG. 16.

FIG. 19 shows a schematic side view of the grid structure of the transducer of FIG. 16.

FIG. 20 shows a cross-sectional view of an alternative embodiment of the double-clamped ultrasonic transducer of the present invention.

FIG. 21 shows a front perspective view of the backplate of FIG. 20.

FIG. 22 shows a back perspective view of the front cover of FIG. 20.

FIG. 23 shows the calculated performance for various dimensions associated with the transducer of FIG. 16 for an 80 KHz acoustic signal.

FIG. 24 is a schematic illustration of the signal flow associated with a digitizer system comprising a transmitter mounted on a movable stylus transmitting ultrasound signals to a transducer receiver according to an aspect of the present invention.

FIG. 25 is a block diagram depicting processing functions associated with the receiving ultrasound transducers according to an aspect of the present invention.

FIG. 26 is an exploded view of the components associated with the transducer of FIG. 16.

FIG. 27 is a front perspective view of transducer element and backplate shown in FIG. 26.

FIG. 28 is a rear perspective view of the assembled components shown in FIG. 27.

FIG. 29 is an exploded view of the components of another embodiment of the double-clamped ultrasonic transducer of the present invention.

FIG. 30A is a perspective view of an embodiment of an embedded ultrasonic micro receiver according to the present invention.

FIG. 30B represents a perspective cut away view of the structure illustrated in FIG. 30A having a protection grid formed thereon.

FIG. 31 is an exploded view of the micro receiver structure of FIG. 30A illustrating the acoustic aperture formed in the exterior surface of the electronic device for receiving the propagating ultrasound signal.

FIG. 32 is a perspective view of the backplate shown in FIG. 31.

FIG. 33A is a perspective view of an alternate embodiment of the embedded ultrasonic micro receiver of the present invention.

FIG. 33B represents a perspective cut away view of the structure illustrated in FIG. 33A having a protection grid formed thereon.

FIG. 34 is an exploded view of the micro receiver structure of FIG. 33A illustrating the acoustic aperture formed in the exterior surface of the electronic device for receiving the propagating ultrasound signal.

FIG. 35A-B represent perspective and cross-sectional views, respectively, of another embodiment of the ultrasonic micro receiver of the invention.

FIGS. 35C-D are cross-sectional and perspective views, respectively, showing the embedded ultrasonic micro receiver of FIGS. 35A-B embodied as a separate, discrete or modular unit.

FIGS. 35E-F are perspective views showing another embodiment of the ultrasonic micro receiver of the present invention embodied as a separate, discrete or modular unit.

FIG. 36 represents a schematic view of another embodiment of the ultrasonic micro receiver of the present invention.

FIG. 37 represents a schematic view of a further embodiment of the ultrasonic micro receiver of the present invention.

FIGS. 38A-B represent perspective and cross-sectional views, respectively, of still another embodiment of the ultrasonic micro receiver of the present invention.

FIGS. 39A-B represent schematic views of another embodiment of the ultrasonic micro receiver of the present invention.

FIGS. 39C-D are schematic views showing the embedded ultrasonic micro receiver of the FIGS. 39A-B embodied as a separate, discrete or modular unit.

FIG. 40 represents a schematic view of yet another embodiment of the ultrasonic micro receiver of the present invention.

FIG. 41A represents a schematic view of another embodiment of embodiment of the ultrasonic micro receiver of the present invention comprising a mono-morph structure.

FIG. 41B is a table including data for the ultrasonic micro receiver of FIG. 41A.

FIGS. 42 and 43A-B represent schematic views of yet another embodiment of the ultrasonic micro receiver of the present invention comprising a capacitive micro-machined ultrasonic transducer structure.

FIGS. 44A-C represent exemplary computer applications where the ultrasonic transducer structures of the present invention can be utilized.

FIGS. 45A-B show an alternative embodiment of the embedded ultrasonic micro receiver of FIGS. 33A-B wherein a recess is provided in the exterior surface of the housing wall section that slopes down to the transducer element.

### DETAILED DESCRIPTION

A first aspect of the present invention is an embedded, ultrasonic transducer (EUT) for hand-held, portable electronic devices of the type including, without limitation, cell phones, PDAs, notebook computers, micro-cassette recorders, and games. The EUT may also be used for other types of electronic devices including, without limitation, keyboards used with personal computers. The EUT is integrated into the housing structure of the electronic device in manner which makes it virtually invisible from an external vantage point and makes it insusceptible to dust and dirt particles.

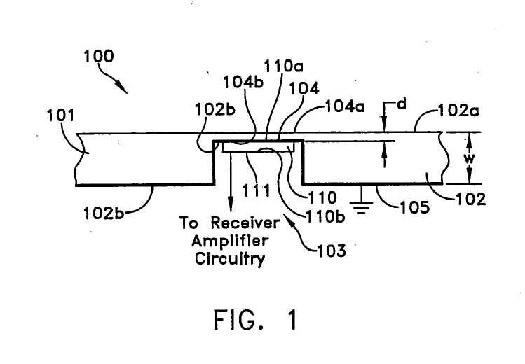

Referring now to the drawings where like parts are indicated with like reference numerals, and initially to FIG. 1, there is shown an embodiment of the EUT of the present invention, denoted by numeral 100. The EUT 100 generally comprises a selected wall section 102 of a housing 101 of an electronic device, and an ultrasonic transducer element 110 embedded in a transducer receiving cavity 103 extending from an interior surface 102b of the selected wall section 102 of the housing 101.

A thin diaphragm 104 is disposed at the bottom of the transducer receiving cavity 103 for operatively supporting the transducer element 110. The diaphragm 104 is unitarily formed with the wall section 102, and has an exterior surface 104a that is flush with an exterior surface 102a of the housing wall section 102. A ground and shielding electrode 105 is disposed on an interior surface 104b of the diaphragm 104, and may substantially cover this surface 104b. The diaphragm 104 has a thickness d that is substantially less than the thickness w of the housing wall section 102, and preferably no more than one-half the thickness w of the wall section 102. This allows the diaphragm 104 to vibrate in response to an impinging acoustic signal applied to its exterior and/or interior surfaces 104a, 104b. In a typical embodiment, the thin diaphragm 104 may have a thickness d of about 0.7 mm.

The earlier-mentioned ground and shielding electrode 105 extends from the interior surface 104b of the diaphragm 104 along a side wall 103a of the transducer receiving cavity 103, to the interior surface 102b of the housing wall section 102. The ground and shielding electrode 105 may then extend a pre-selected distance along interior surface 102b of the wall section 102.

The transducer element 110 may comprise a thin film of piezoelectric material including, without limitation, polyvinylidene fluoride (PVDF). A PVDF-based transducer element 110 typically has a thickness of about 110 microns (82 m). A working electrode 111 is disposed on an interior facing surface 110b of the transducer element 110, and may substantially cover this surface 110b. (The term “working electrode,” as used herein, refers to an electrode which allows the transducer element to electrically communicate with receiving or transmitting circuitry of the electronic device.) The diaphragm facing surface 110a of the transducer element 110 is adhesively bonded to the interior surface 104b of the diaphragm 104. When the length of the transducer element 110 (the thin film of piezoelectric material) expands or shrinks by external force, it developes a voltage on the surface electrodes 105, 111. This length-wise strain in the transducer element 110 is caused by flexural motion of the diaphragm 104. Therefore, vibration of the diaphragm 104 generates a voltage which is fed to the receiver circuitry. An epoxy or other suitable adhesive bonding material, may be used for adhesively bonding the transducer element 110 to the diaphragm 104.

As shown in the embodiment of FIG. 1, the exterior surface of the housing wall section 102 is substantially flat or planar. The diaphragm 104 and the transducer element 110 are substantially flat or planar and preferably circular in plan, and the transducer receiving cavity 103 is preferably cylindrical. The diameter of the preferred circular transducer element 110 is typically about 8 mm for a 40 KHz frequency EUT 100 and about 5.7 mm for an 80 KHz frequency EUT 100, using the typical diaphragm and transducer thicknesses provided above. It should be noted that the square of the diameter of the transducer element 110 is generally inversely proportional to the resonance frequency.

The housing wall section 102 and the diaphragm 104 are typically made from the same material used for making the housing 101 of the electronic device, as the housing wall section 102 is usually formed unitary with the housing 101 of the electronic device. Such materials include, without limitation, electrically insulative materials, such as plastic, which can be formed using any conventional plastic forming method, such as plastic injection molding. It is contemplated that the housing wall section 102 (and diaphragm 104) of the EUT 100 may also be formed as a separate, discrete unit and combined with the rest of the housing 101.

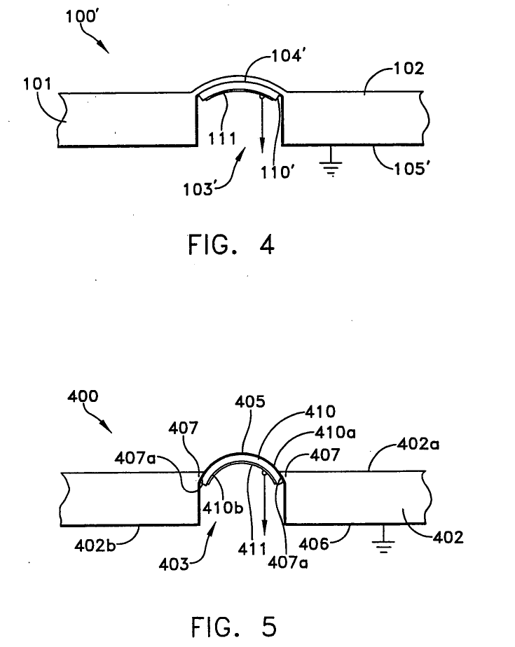

One of ordinary skill in the art will recognize that the housing wall section 102, the transducer receiving cavity 103, the diaphragm 104, and the ultrasonic transducer element 110 of the EUT 100 embodied in FIG. 1, may have other geometrical shapes. For example, FIG. 4 shows an EUT 100′ comprising a rectangular or square, outwardly curved diaphragm 104′ disposed at the bottom of a corresponding rectangular or square transducer receiving cavity 103′, and a rectangular or square, outwardly curved ultrasonic transducer element 110′. The curved diaphragm 104′ behaves differently from a flat diaphragm, in that it does not flex. Instead, the incoming acoustic pressure generates a strain in the plane of the curved direction because the periphery of the curved diaphragm is fixed, therefore, a voltage is generated. The resonance condition is also different. The curved diaphragm 104′ has a resonance frequency given by approximately F₀=200/R where R is the radius of the curvature (in meters). In one embodiment, a 40 KHz EUT 100′ can be fabricated using a curved diaphragm 110′ having a radius of about 5 mm. An 80 KHz EUT 100′ can be fabricated using a curved diaphragm 110′ having a radius of about 2.5 mm. The influence of the thickness of the diaphragm to the resonance frequency is negligible as this is a unique feature of curved film resonators.

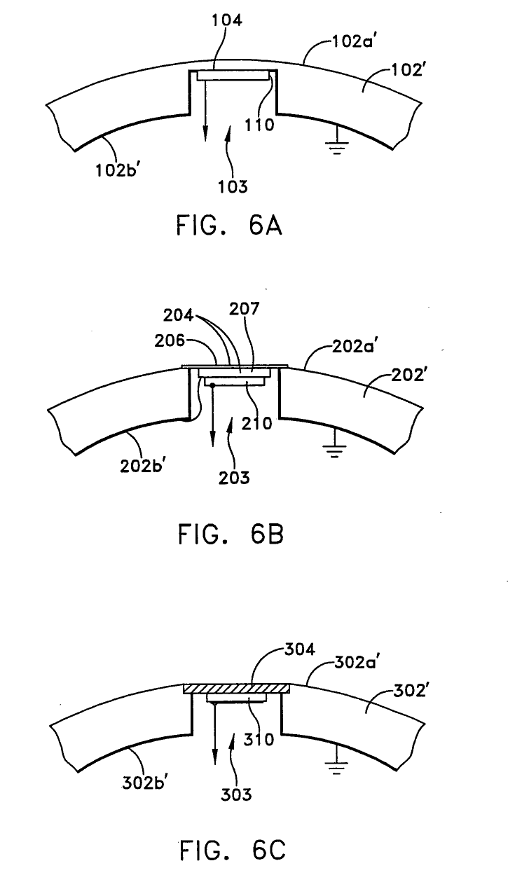

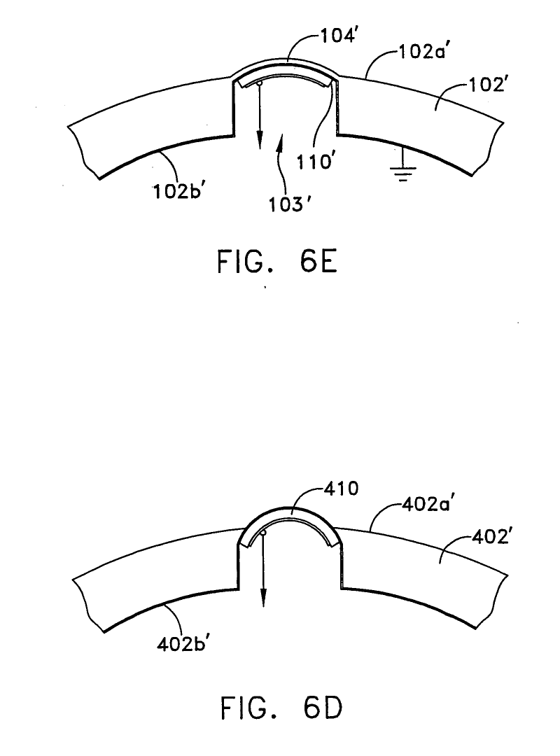

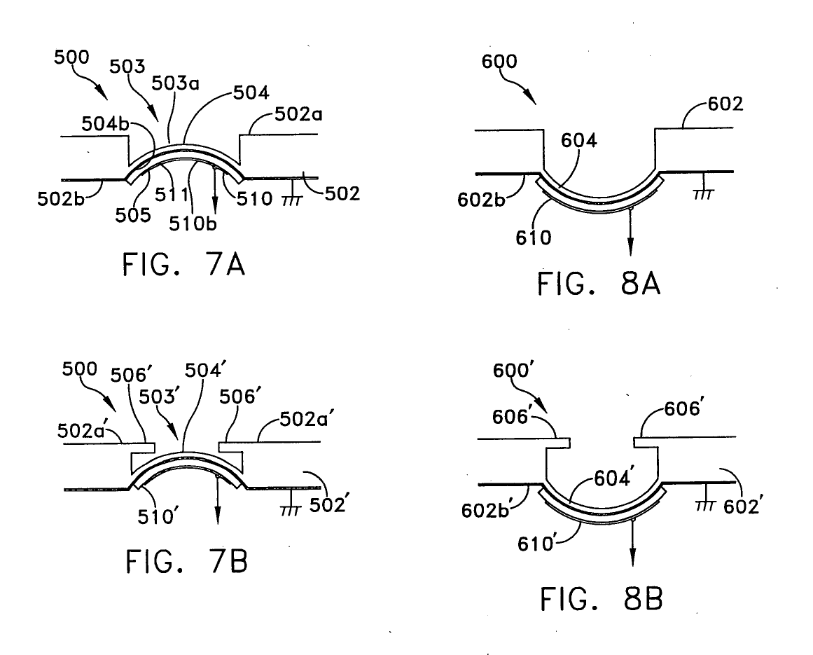

The transducer receiving cavity 103, the diaphragm 104, and the transducer element 110 of FIG. 1 can be utilized, as shown in FIG. 6A, with a curved housing wall section 102′ that has curved exterior and interior surfaces 102a′,102b′. The transducer receiving cavity 103′, the diaphragm 104′, and the transducer element 110′ of FIG. 4 can also be utilized with a curved housing wall section 102′ having curved exterior and interior surfaces 102a′,102b′, as shown in FIG. 6E.

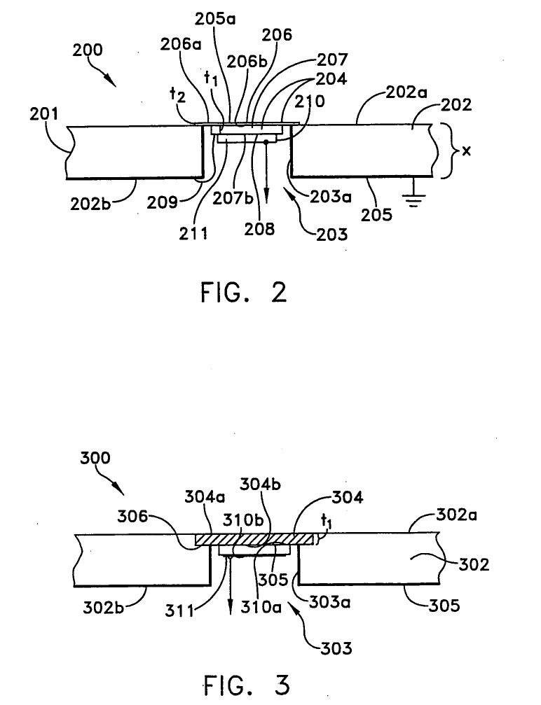

FIG. 2 shows another embodiment of the EUT of the present invention. The EUT, denoted by numeral 200, is similar to the embodiments of FIGS. 1, 4, 6A, and 6E in that it generally comprises a selected wall section 202 of a housing 201 of an electronic device, and an ultrasonic transducer element 210 embedded in a transducer receiving cavity 203 extending from an interior surface 202b of the selected wall section 202 of the housing 201. However, the bottom of the transducer receiving cavity 203 is formed in the embodiment of FIG. 2 by a supporting diaphragm structure 204 comprising a very thin outer film 206 that is separately attached to an exterior surface 202a of the wall section 202 by an adhesive, for example, and an inner film 207 that is adhesively bonded to an interior surface 206b of the outer film 206. The outer film 206 may be made of a stainless steel material, and may have a thickness d₁ of about 50 microns, which allows an exterior surface 206a of the outer film 206 to be substantially flush with an exterior surface 202a of the wall section 202. The inner film 207 may be made of a non-piezoelectric, polymeric material, such as polyester, and may having a thickness d₂ of about 250 microns.

A shielding and ground electrode 205 extends a pre-selected distance along interior surface 202b of the wall section 202, and along a side wall 203a of the transducer receiving cavity 203. The shielding and ground electrode 205 includes a shielding electrode portion 205a disposed on the interior surface 206b of the outer film 206, which may substantially cover this surface 206b. A ground electrode portion 208 is disposed on the interior surface 207b of the inner film 207, which may substantially cover this surface 207b. The ground electrode portion 208 is electrically coupled to either the shielding electrode portion 205a or the shielding and ground electrode 205. Electrically coupling may be implemented with a mechanical pressure contact 209 or other means.

The transducer element 210 may comprise a thin film of piezoelectric material including, without limitation, PVDF. A PVDF-based transducer element 210 typically has a thickness of about 110 μm. A working electrode 211 is disposed on an interior facing surface 210b of the transducer element 210, and may substantially cover this surface 210b. The diaphragm facing surface 210a of the transducer element 210 is adhesively bonded to the interior surface 207b of the inner film 207. The principle voltage generation is the same as the embodiment of FIG. 1. However, there is a difference in the enhancement of the output because a larger strain in the transducer element 210 (PVDF) is developed by the thicker structure due to inner film 207. An epoxy or other suitable adhesive bonding material, may be used for adhesively bonding the transducer element 210 to the inner film 207.

As shown in the embodiment of FIG. 2, the exterior surface of the housing wall section 202 is substantially flat or planar. The outer and inner films 206, 207 of the diaphragm structure 204 and the transducer element 210 are substantially flat or planar and preferably circular in plan, and the transducer receiving cavity 203 is preferably cylindrical. The diameter of the preferred circular transducer element 210 is typically about 5.4 mm for a 40 KHz frequency EUT 200 and about 3.8 mm for an 80 KHz frequency EUT 200, using the typical inner film and transducer thicknesses provided above. Increasing the thickness of the transducer element 210 requires a corresponding reduction in the thickness of the inner film 207 in order to keep the resonance frequency substantially the same. Increasing the thickness of the transducer element 210, increases the acoustic sensitivity of the EUT 200.

The housing wall section 202 is typically made from the same material used for making the housing 201 of the electronic device, as the housing wall section 202 is usually formed unitary with the housing 201 of the electronic device. Such materials include, without limitation, electrically insulative materials, such as plastic, which can be formed using any conventional plastic forming method, such as plastic injection molding. It is contemplated that the housing wall section 202 of the EUT 200 may also be formed as a separate, discrete unit and combined with the rest of the housing 201.

One of ordinary skill in the art will recognize that the housing wall section 202, the transducer receiving cavity 203, the outer and inner films 206, 207 of the diaphragm structure 204, and the ultrasonic transducer element 210 of the EUT 200 embodied in FIG. 2, may have other geometrical shapes. For example, the transducer receiving cavity 203, the outer and inner films 206, 207 of the diaphragm structure 204, and the transducer element 210 of FIG. 2 can be utilized, as shown in FIG. 6B, with a curved housing wall section 202′ having curved exterior and interior surfaces 202a′,202b′.

FIG. 3 shows yet another embodiment of the EUT of the present invention. The EUT, denoted by numeral 300, is similar to the embodiments of FIGS. 1 and 2, except that the EUT 300 comprises a plate-like diaphragm 304 disposed at the bottom of the transducer receiving cavity 303. The marginal periphery portion of the diaphragm 304 is seated in a recess 306, formed in the exterior surface 302a of the wall section 302, surrounding the transducer receiving cavity 303. An adhesive may be used for retaining the diaphragm in the recess 306. A ground and shielding electrode 305 is disposed on an interior surface 304b of the plate-like diaphragm 304, and may substantially cover the portion of this surface 304b facing the inside of the transducer receiving cavity 303. The plate-like diaphragm 304 may be made of a metallic material, such as aluminum or stainless steel, and may have a thickness d of about 0.6 mm. The depth of the recess 306 and the thickness of the plate-like diaphragm 304 are preferably the same so that the outer surface 304a of the diaphragm 304 lies substantially flush with the exterior surface 302a of the wall section 302.

The earlier-mentioned ground and shielding electrode 305 extends from the interior surface 304b of the plate-like diaphragm 304 along a side wall 303a of the transducer receiving cavity 303, to the interior surface 302b of the housing wall section 302. The ground and shielding electrode 305 may then extend a pre-selected distance along interior surface 302b of the wall section 302.

The ultrasonic transducer element 310 may comprise a thin film of piezoelectric material including, without limitation, lead-zirconate-titanate (PZT). A PZT-based transducer element 310 typically has a thickness of about 300 μm. A working electrode 311 is disposed on an interior facing surface 310b of the transducer element 310, and may substantially cover this surface 310b. The diaphragm facing surface 310a of the transducer element 310 is adhesively bonded to the interior surface 304b of the plate-like diaphragm 304 to ensure proper mechanical stressing of the transducer element 310 in response to an acoustic input applied thereto. An epoxy or other suitable adhesive bonding material, may be used for adhesively bonding the transducer element 310 to the plate-like diaphragm 304.

As shown in the embodiment of FIG. 3, the exterior surface of the housing wall section 302 is substantially flat or planar. The plate-like diaphragm 304 and the transducer element 310 are substantially flat or planar and preferably circular in plan, the transducer receiving cavity 303 is preferably cylindrical. The diameter of the preferred circular transducer element 310 is typically about 10 mm for a 40 KHz frequency EUT 300 and about 7 mm for an 80 KHz frequency EUT 300, using the typical diaphragm and transducer thicknesses provided above. The thickness combination of the diaphragm 304 and transducer element 310 may be different than disclosed above, and resonance frequency is also different. If each thickness in the layer structure is different by a factor of N the diameter of the transducer element 310 is proportional to the square root of N to keep the resonance frequency constant such that the thicker the material, the larger the diameter.

The housing wall section 302 is typically made from the same material used for making the housing 301 of the electronic device, as the housing wall section 302 is usually formed unitary with the housing 301 of the electronic device. Such materials include, without limitation, electrically insulative materials, such as plastic, which can be formed using any conventional plastic forming method, such as plastic injection molding. It is contemplated that the housing wall section 302 of the EUT 300 may also be formed as a separate, discrete unit and combined with the rest of the housing 301.

One of ordinary skill in the art will recognize that the housing wall section 302, the transducer receiving cavity 303, the plate-like diaphragm 304, and the transducer element 310 of the EUT 300 embodied in FIG. 3, may have other geometrical shapes. For example, the transducer receiving cavity 303, the diaphragm 304, and the transducer element 310 of FIG. 3 can be utilized, as shown in FIG. 6C, with a curved housing wall section 302′ having curved exterior and interior surfaces 302a′,302b′.

FIG. 5 shows a further embodiment of the EUT of the present invention, denoted by numeral 400. The EUT 400 comprises a rectangular or square, outwardly curved ultrasonic transducer element 410 mounted at the bottom of a corresponding rectangular or square transducer receiving cavity 403. No diaphragm structure is utilized in this embodiment.

The transducer element 410 may comprise a thin film of piezoelectric material including, without limitation, polyvinylidene fluoride (PVDF). A PVDF-based transducer element 410 typically has a thickness of about 28-110 μm. A ground and shielding electrode portion 405 is disposed on an exterior surface 410a of the transducer element 410, and a working electrode 411 is disposed on the interior surface 410b of the transducer element 410. The electrodes 405, 411 may substantially cover these surfaces 410a, 410b. The ground and shielding electrode 405 portion communicates with a ground and shielding electrode 406 which extends along a side wall 403a of the transducer receiving cavity 403, to the interior surface 402b of the housing wall section 402. The ground and shielding electrode 405 may then extend a pre-selected distance along interior surface 402b of the wall section 402.

The ends of the transducer element 410 are adhesively bonded (clamped) to two radial inwardly projecting mounting flanges 407 disposed at the bottom of the transducer receiving cavity 403. The mounting flanges 407 each have a curved mounting surface 407a that defines the desired curvature of the transducer element 410. This method of mounting ensures that the transducer element 410 is formed with a curvature so that it generates a voltage in response to an acoustic input applied thereto. An epoxy or other suitable adhesive bonding material, may be used for adhesively bonding the transducer element 410 to the mounting surface 407a of the flange 407.

As shown in the embodiment of FIG. 5, the exterior surface of the housing wall section 402 is substantially flat or planar. One of ordinary skill in the art will recognize, however, that a curved housing wall section 402′, having curved exterior and interior surfaces 402a′,402b′ can also be utilized in this embodiment as shown in FIG. 6D.

The housing wall section 402 is typically made from the same material used for making the housing 401 of the electronic device, as the housing wall section 402 is usually formed unitary with the housing 401 of the electronic device. Such materials include, without limitation, electrically insulative materials, such as plastic, which can be formed using any conventional plastic forming method, such as plastic injection molding. It is contemplated that the housing wall section 402 of the EUT 400 may also be formed as a separate, discrete unit and combined with the rest of the housing 401.

FIG. 7A shows an additional alternative embodiment of the EUT of present invention. The EUT of FIG. 7A, denoted by numeral 500, is substantially identical to the EUT 100′ of FIG. 4 except, that the rectangular or square, outwardly curved diaphragm 504 is recessed from the exterior surface 502a of the housing wall section 502 such that the apex 504c of the diaphragm's exterior surface 504a is withdrawn from the exterior surface 502a of the housing wall section 502. This recessed structure enables the transducer element 510 to be better protected from external forces, which may inadvertently impact the device. The transducer receiving cavity 103′ of FIG. 4 is now an acoustic aperture 503 having an opening 503a in this embodiment. The interior surface 504b of the diaphragm 504 includes a shielding and ground electrode 505 that extends to the interior surface 502b of the housing wall section 502, and supports a corresponding rectangular or square, outwardly curved ultrasonic transducer element 510, which is adhesively bonded thereto. The interior surface 510b of the transducer element 510 includes a working electrode 511.

Generally, angle performance or directivity of the sensitivity of the transducer (maximum at normal incidence and weaker at angled incidence) shows a broader range of the high sensitivity region when the aperture is smaller. In other words, the directionality of the EUT 500 can be widened by narrowing the opening 503a of the acoustic aperture 503. This can be accomplished, as shown in FIG. 7B, by providing inwardly directed flanges 506′ having exterior surfaces 506a′ which may be flush with the exterior surface 502a′ of the housing wall section 502′. The flanges 506′ also aid in protecting the transducer element 510′.

The diaphragms 504, 504′ and the transducer elements 510, 510′ of the embodiments of FIGS. 7A and 7B, each curve toward the exterior surface 502a, 502a′ of its respective housing wall section 502, 502′. However, FIGS. 8A and 8B are embodiments of EUTs 600, 600′ with diaphragms 604, 604′ and transducer elements 610, 610′ that each curve toward the interior surface 602b, 602b′ of its respective housing wall section 602, 602′.

FIG. 9A shows still another embodiment of the EUT of present invention. The EUT of FIG. 9A, denoted by numeral 700, is substantially identical to the EUT 400 of FIG. 5 except, that that the rectangular or square, outwardly curved transducer element 710 is recessed from the exterior surface 702a of the housing wall section 702 such that the apex 710c of the transducer elements exterior surface 710a is withdrawn from the exterior surface 702a of the housing wall section 702. This recessed structure enables the transducer element 710 to be better protected from external forces, which may inadvertently impact the device. The transducer receiving cavity 403 of FIG. 5 is now an acoustic aperture 703 having an opening 703a in this embodiment. The exterior surface 710a of the transducer element 710 includes a shielding and ground electrode 705 that extends to the interior surface 702b of the housing wall section 702. The interior surface 710b of the transducer element 710 includes a working electrode 711.

Another difference is the use of a back plate 712 to clamp the ends 710c of the transducer element 710. The surface 712a of the back plate 712 that faces the transducer element 710, includes two angled clamping surfaces 712b disposed inwardly from opposing ends 712c of the back plate 712. Buffers 713 made from a resilient material, such as rubber for example, may be disposed between the clamping surfaces 712b of the backplate 712 and the ends 710c of the transducer element 710 to clamp the ends 710c of the transducer element 710 to the radial inwardly projecting mounting flanges 707 disposed at the bottom of the acoustic aperture 703. The mounting flanges 707 have curved mounting surfaces 707a that define the desired curvature of the transducer element 710. This method of mounting ensures that the transducer element 710 is mechanically fixed in a manner which forms a predetermined curvature and causes it to generate a voltage in response to an acoustic input applied thereto. The backplate 712 includes an aperture 712c for providing electrical connectivity to the working electrode disposed on the interior surface 710b of the transducer element 710. The ends of the backplate 712 are conventionally adapted includes to snap fit into corresponding grooves defined in the interior surface 702b of the housing wall section 702.

A ground and shielding electrode portion 705 is disposed on an exterior surface 710a of the transducer element 710. The ground and shielding electrode 705 portion communicates with a ground and shielding electrode 706 which extends along the interior surface 702b of the housing wall section 702.

The directionality of the EUT 700 can be widened by narrowing the opening 703a of the acoustic aperture 703 as mentioned above. This can be accomplished, as shown in the embodiment of FIG. 9B, by providing an inwardly directed flanges 706′ having exterior surfaces 706a′ which may be flush with the exterior surface 702a′ of the housing wall section 702′. The flanges 706′ also aid in protecting the transducer element 710′.

The transducer elements 710, 710′ of the embodiments of FIGS. 9A and 9B each curve toward the exterior surface 702a, 702a′ of its respective housing wall section 702, 702′. However, FIGS. 10A and 10B are embodiments showing EUTs 800, 800′ with transducer elements 810, 810′ that each curve toward the interior surface 802b, 802b′ of its respective housing wall section 802, 802′.

As shown in the embodiment of FIG. 11, a protective layer 714 can be provided on the exterior surface 710a of the transducer element 710 to further aid in protecting shielding and ground electrode portion 705, which may become damaged over time by contact. The protection layer 714 may comprise, for example, a 25 um polyester or polyimid layer, which may be adhesively bonded to the electrode portion 705.

As further shown in FIG. 11, the transducer element 710 can be further protected by a protective cover 716 formed as a wire mesh or grid, for example, which is disposed over the acoustic aperture 703 and coupled at opposite ends thereof via retaining members 717 secured to the exterior surface 702a of the housing wall section 702.

FIG. 12 shows another embodiment of the EUT of the present invention, denoted by numeral 700″, having a rectangular or square, outwardly curved ultrasonic transducer element 710″ spaced from the interior surface 702b″ of the housing wall section 702.″ The transducer element 710″ is adhesively bonded to interiorly projecting mounting flanges 707″ depending from the interior surface 702b″ of the housing wall section 702.″ The mounting flanges 707″ each have a curved mounting surface 707a″ that defines the desired curvature of the transducer element 710.″ Only the ends 710c″ of the transducer element 710″ is bonded or clamped to the mounting surfaces 707a″ of the flanges 707.″ This method of mounting ensures that the transducer element 710″ is mechanically fixed in a manner which forms a predetermined curvature and causes it to generate a voltage in response to an acoustic input applied thereto. An epoxy or other suitable adhesive bonding material, may be used for adhesively bonding the transducer element 710″ to the mounting surface 707a″ of the flange 707.″ The space between the side walls 707b″ of the mounting flanges forms an acoustic aperture 703″ with a narrowed opening 703a″ of any desired geometrical shape including, without limitation, a circular, square, or rectangular shape.

Disposed on the aperture facing surface 710a″ of the transducer element 710″ is a shielding and ground electrode 705″, which may substantially cover this surface 710a.″ A working electrode 711″ is disposed on the interior surface 710b″ of the transducer element 710.″ The working electrode 711″ may also substantially cover the interior surface 710b″ of the transducer element 711.″

The top view of FIG. 13 and the side view of FIG. 14 collectively show still another embodiment of the EUT of the present invention, denoted by numeral 900. In this embodiment, an omnidirectional ultrasonic transducer 910 is suspended from an interior surface 902b of a housing wall section 902 of a housing 901 of an electronic device, on a support extension 904. The transducer 910 includes a spool-shape main body 910a, and an unclamped cylindrical transducer element 910b, which may comprise a thin film of piezoelectric polymer material (e.g. PVDF), disposed about the main body 910a and resting on a radial outwardly extending flange 910c. An air gap g is formed between an axle portion 910d of the main body 910a and the transducer element 910b. The interior cylindrical surface 910e of the transducer element 910b has disposed thereon a working electrode 911, and the exterior cylindrical surface 910f of the transducer element 910b has disposed thereon a shielding and ground electrode 905. Such a transducer is disclosed in U.S. Pat. No. 6,411,014 entitled “Cylindrical Transducer Apparatus.” As shown, a cylindrical surface portion of the transducer element 910b faces an opening 903 in the housing wall section 902, which allows the passage therethrough of acoustic signals.

One or more of the earlier described EUTs of the present invention may be integrated into the electronic device, depending upon the application. For example, FIG. 15 shows a schematic view wherein at least two EUTs 150 are integrated into a portable electronic device 120, such as a wireless telephone. The telephone 120 may include a housing 121 which includes a display area 121a, a keypad 121b, an audio input 121c and an audio output 121d. The EUTs 150 may be integrated in the side of the telephone housing 121 at predetermined distance from one another. One or both of the EUTs 150 may be used as a receiver to detect incoming acoustic signals generated by a remotely located ultrasonic transducer or as a transmitter to generate acoustic signals which are detected by the remotely located ultrasonic transducer. As a receiver, the EUT detects an incoming acoustic signal impinging the diaphragm. As shown in FIG. 15, ultrasound energy signals 130 and 131 radiated from an ultrasonic transducer 141 mounted on a movable stylus 140 impinge the diaphragms (or the transducer elements directly) and cause them to vibrate. The vibrations may be converted to an electrical signal by the transducer element (not shown) of each EUT 150, which may be processed via electronics (not shown) in conventional fashion. Accordingly, the electrical signal generated by the transducer element may be used to indicate the acoustic waveform of the impinging acoustic signal.

As a transmitter, the application of a voltage to the transducer element of each EUT 150 will cause it to vibrate. The vibrations radiate an acoustic signal in the direction substantially normal to the exterior surface of the diaphragm (or transducer element).

A second aspect of the present invention is an embedded ultrasonic micro receiver (EUMR) for hand-held, portable electronic devices of the type including, without limitation, cell phones, PDAs, notebook computers, micro-cassette recorders, and games. This aspect of the invention is characterized by a very narrow width (1-2 mm) transducer element or film and a very small device structure. The purpose of these features is to make the EUMR invisible while substantially maintaining sensitivity, which is 50-70% of a larger size receiver. The EUMR may also be used for other types of electronic devices including, without limitation, keyboards used with personal computers. The EUMR may be integrated into the housing structure of the electronic device in manner which makes it virtually invisible from an external vantage point and makes it insusceptible to dust and dirt particles. The EUMR receives acoustic signals propagating along a surface S of the electronic device.

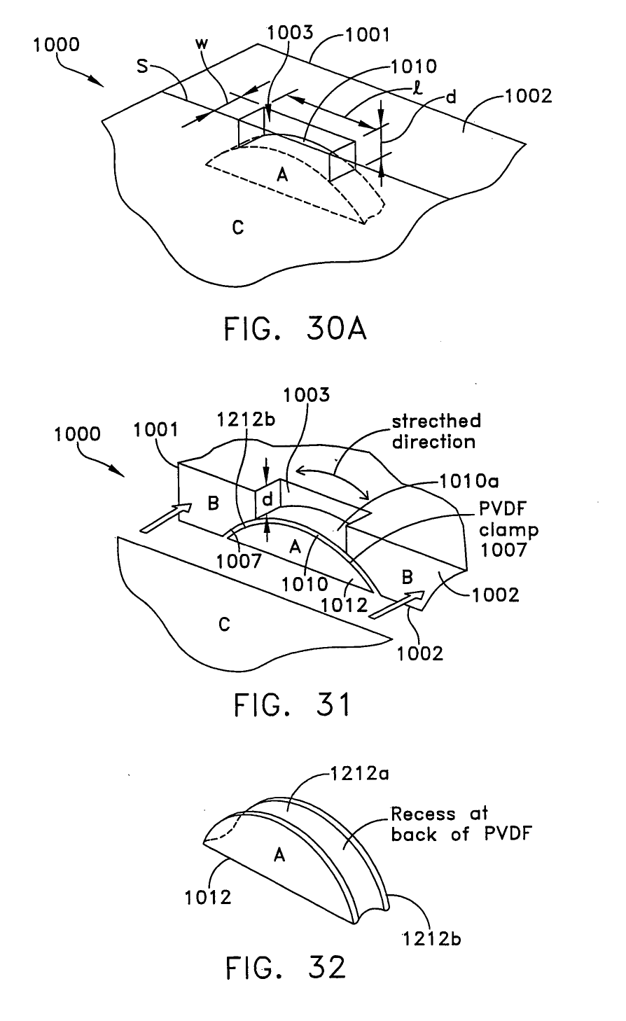

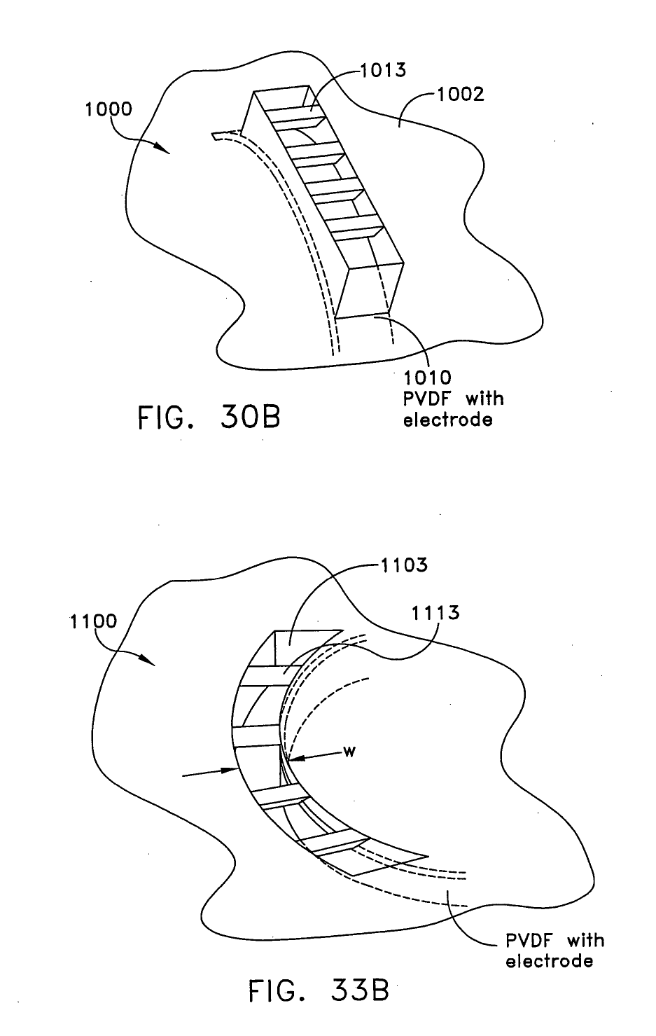

Referring again to the drawings where like parts are indicated with like reference numerals, and initially to FIGS. 30A-B, 31, and 32, there is shown an embodiment of the EUMR of the present invention, denoted by numeral 1000. The EUMR 1000 generally comprises a selected wall section 1002 of a housing 1001 of an electronic device, and a outwardly curved ultrasonic transducer element 1010 clamped to an interior surface 1002b of the selected wall section 1002 and partially disposed within a very small rectangular acoustic aperture 1003 extending through the housing wall section 1002 of the device. The transducer element 1010 is positioned such that an exterior surface 1010a of the transducer element 1010 generally faces the acoustic aperture 1003.

The transducer element 1010 is clamped to the interior surface 1002b of the housing wall section 1002 by a back plate 1012, which is shown alone in FIG. 32. The back plate 1012 includes an outwardly curved, V-shape surface 1012a, which defines a pair of outwardly curved edges 1012b. The backplate 1012 may be conventionally adapted to snap fit against or into the interior surface 1002b of the housing wall section 1002.

The transducer element 1010 may comprise a thin, rectangular film of piezoelectric material including, without limitation, a thin, rectangular film of PVDF which has been longitudinally stretched. A PVDF-based transducer element 1010 typically has a thickness of about 28 μm. A ground and shielding electrode (not shown) may be disposed on the exterior surface 1010a of the transducer element 1010, and a working electrode (not shown) may be disposed on the interior surface 1010b of the transducer element 1010. The electrodes may substantially cover the transducer surfaces 1010a, 1010b.

The ends of the transducer element 1010 are clamped between two inwardly curved clamping surfaces 1007 defined in the interior surface 1002 of the housing wall section 1002, at each end of the acoustic aperture 1003, and the outwardly curved edges 1012a of the back plate 1012. The curved clamping surfaces and edges 1007, 1012b define the desired curvature of the transducer element 1010. This method of mounting ensures that the transducer element 1010 is mechanically fixed at two ends in a manner which causes it to generate a voltage in response to an acoustic input applied thereto, as discussed earlier with respect to FIGS. 4 and 5. Note that the EUMR 1000 can also be formed with a protection grid 1013 across the acoustic aperture 1003 as shown in FIG. 30B. The grid 1013 may provide a minor obstruction to a propagating waveform, however, a suitable ratio can be established between the grid 1013 and the size of the acoustic aperture 1003, which will maximize the sensitivity of the EUMR 1000.

The apex of transducer element 1010 of the EUMR shown in FIGS. 30A-B, 31, and 32, may be disposed in the acoustic aperture 1003 at a depth d (FIGS. 30A-B) of less than 1 mm from an exterior surface 1002 of the housing wall section 1002. The acoustic aperture 1003 may have a width w between 1 mm and 2 mm and a length 1 between 2.5 mm and 4.0 mm for an 80 KHz EUMR. The sensitivity is about 80% at d=0 mm due to wave propagation being parallel to the transducer film plane. When d=1 mm, the signal is reduced to about 20%-40%. Herein, 100% means the sensitivity when the acoustic wave is incident perpendicularly to the surface at the apex.

The housing wall section 1002 is typically made from the same material used for making the housing 1001 of the electronic device, as the housing wall section 1002 is usually formed unitary with the housing 1001 of the electronic device. Such materials include, without limitation, electrically insulative materials, such as plastic, which can be formed using any conventional plastic forming method, such as plastic injection molding. It is contemplated that the housing wall section 1002 of the EUMR 1000 may also be formed as a separate, discrete unit and combined with the rest of the housing 1001.

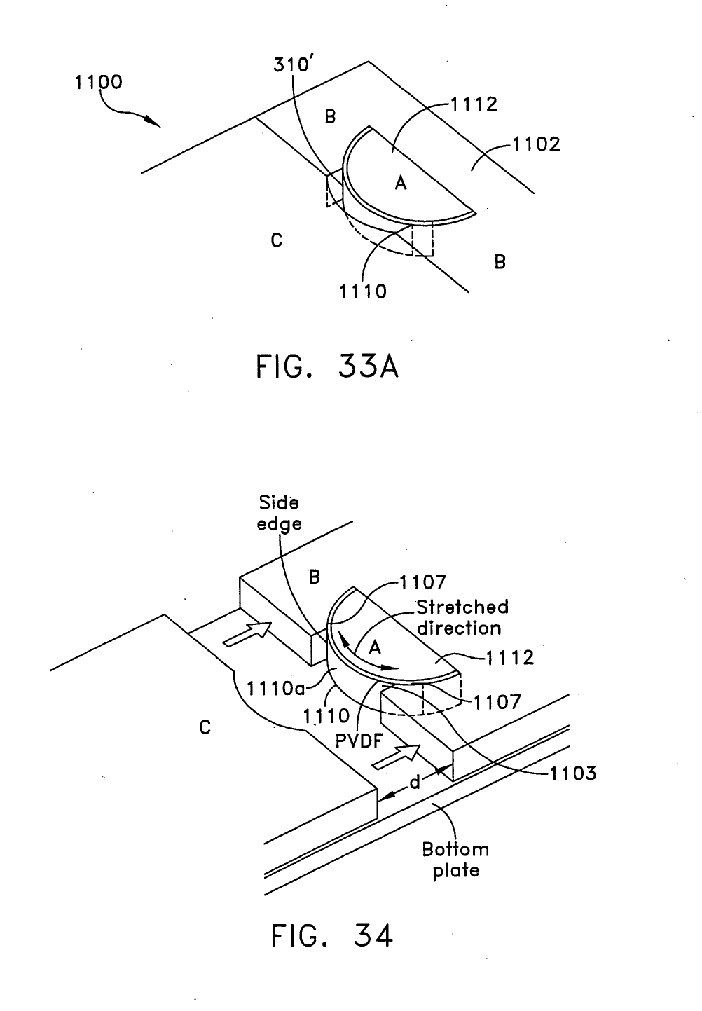

FIGS. 33A-B, and 34, show another embodiment of the EUMR of the present invention, denoted by numeral 1100. The EUMR 1100 of this embodiment is very similar to the EUMR 1000 of FIGS. 30A-B, 31, and 32 except, that EUMR 1100 includes a transducer element 1110 and back plate 1112 assembly which is oriented sideways or horizontally to the housing wall section 1102 (as compared with vertical or upright to the housing wall section as in FIGS. 30A-B, 31, and 32). In addition, the EUMR 1100 includes a curved acoustic aperture 1103 that has a curvature which is generally identical to that of the curved transducer element 1110. This design positions an exterior surface 1110a of the transducer element orthogonal to the acoustic aperture 1103.

In addition, curved clamping surfaces 1107 start at side edges of the acoustic aperture 1103, adjacent the ends thereof instead of at bottom edges of the acoustic aperture 1003, as in FIGS. 30A-B, 31, and 32.

In the transducer element 1110 of the EUMR shown in FIGS. 33A-B and 34, the propagation direction for the highest sensitivity is generally perpendicular to the transducer element's exterior surface 1110a surface at the center thereof when the width w of the acoustic aperture 1103 becomes very large, the sensitivity reaching a maximum value when the depth d=0 mm. As obstructing surface C approaches such that width w of the acoustic aperture 1103 is between 0.5 mm and 1.0 mm, the sensitivity diminishes to about 20%. A further decrease in width w (w=0.1 mm-0.3 mm) of the acoustic aperture 1103 causes the signal to increase to 40-50%. In this case the depth d is substantially constant and about equal to the width of the film. Note that as in the previous embodiment, the EUMR 1100 can also be formed with a protection grid 1113 disposed across the acoustic aperture 1103, as shown in FIG. 33.

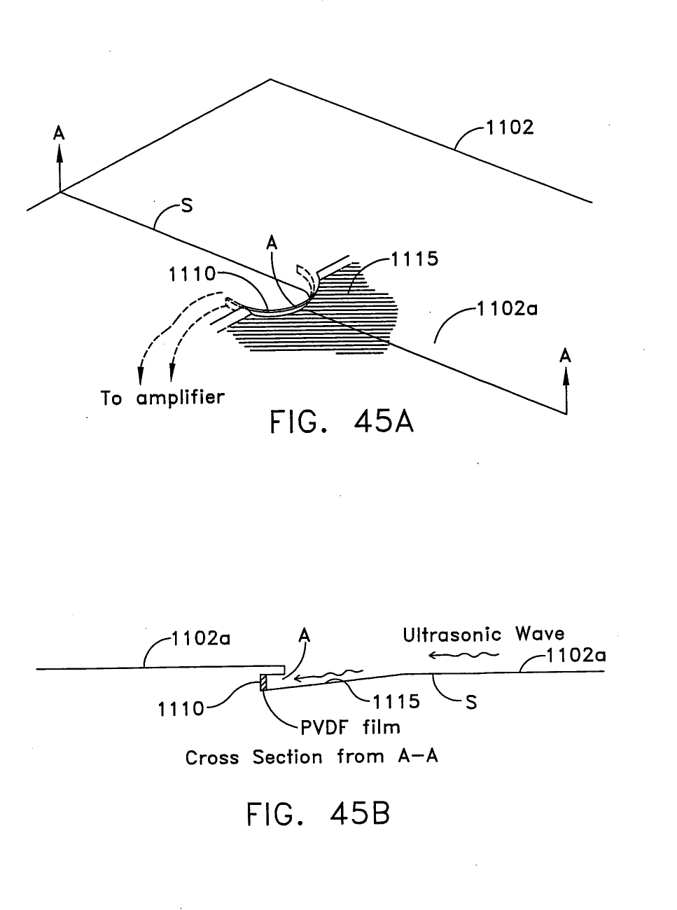

As shown in FIGS. 45A-B, the outwardly curved side wall of the acoustic aperture of the embodiment shown in FIGS. 33A-B and 34 can be replaced with a recess 1115 that slopes progressively down from the exterior surface 1102a of the housing wall section 1102 to the transducer element 1110. In this embodiment, the plane of the transducer element 1110 is perpendicular to an acoustic wave, the wave propagating along the surface of the recess 1115. The EUMR of this embodiment has a sensitivity which is about twice as great as the EUMR embodied in FIGS. 33A-B and 34.

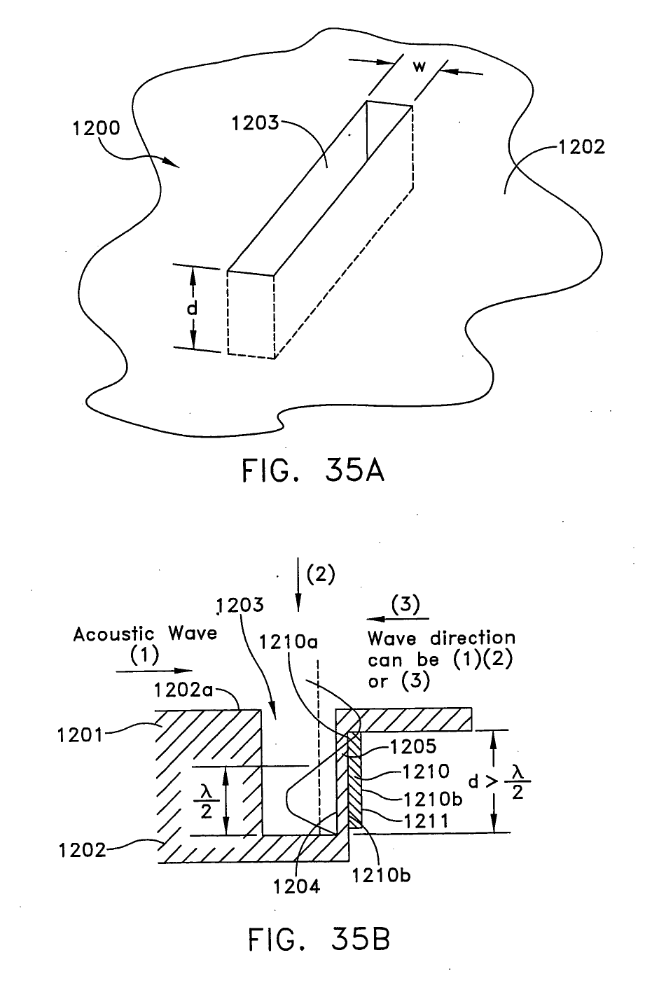

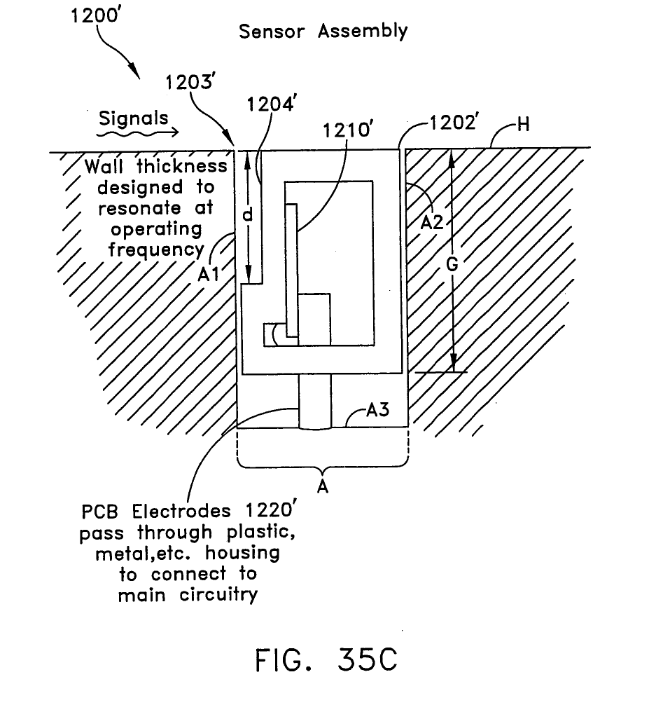

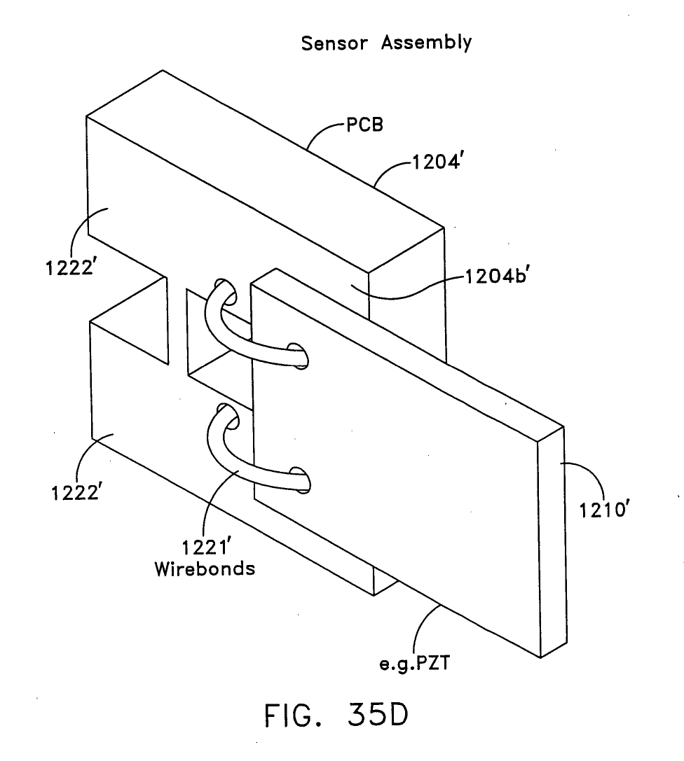

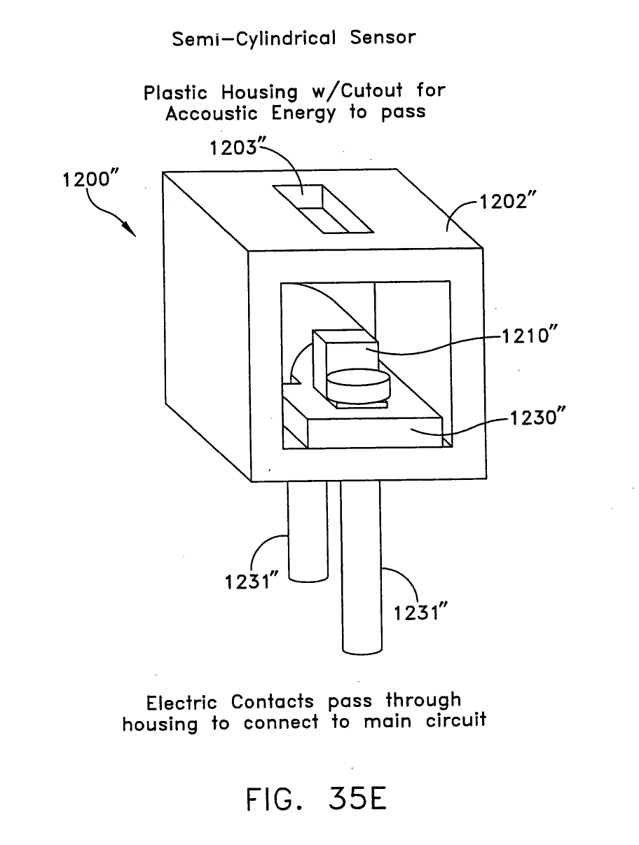

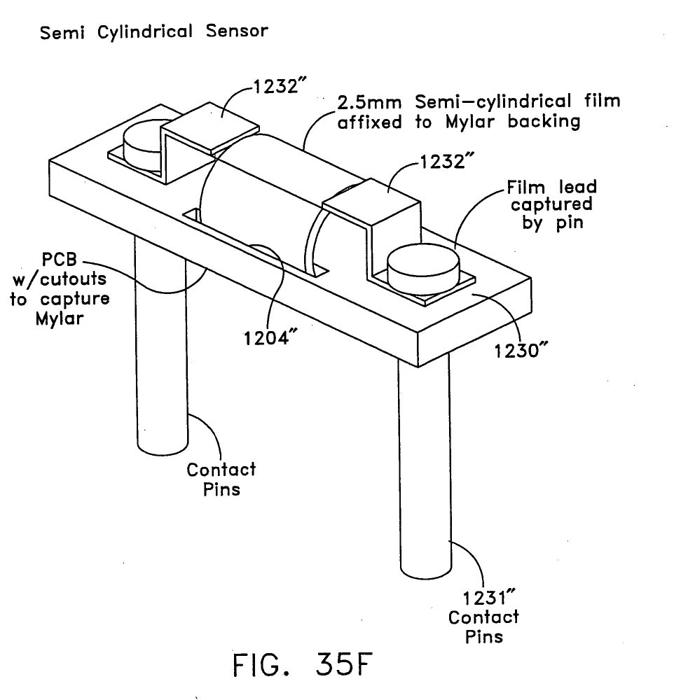

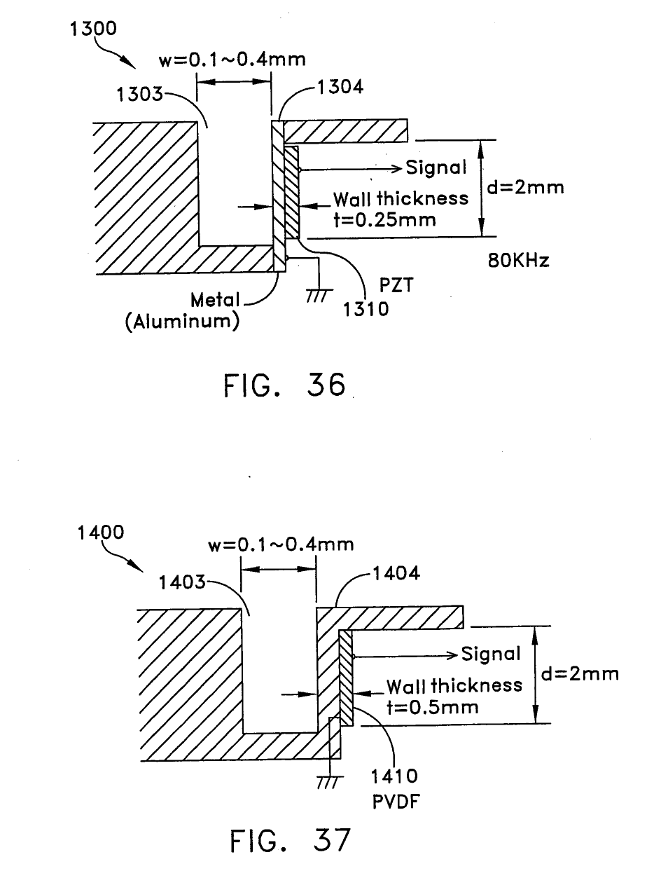

FIGS. 35A-B, show yet another embodiment of the EUMR of the present invention, denoted by numeral 1200, the specific details of which will be described further on with reference to FIGS. 36 and 37. The EUMR 1200 generally comprises a selected wall section 1202 of a housing 1201 of an electronic device, and a narrow acoustic aperture 1203 of width w and depth d formed in a substantially flat, exterior surface 1202a of the housing wall section 1202. The acoustic aperture 1203 has a substantially planar side wall 1204 extending perpendicular to the exterior surface 1202a of the housing wall section 1202 and unitary with the wall section, which is operative as a diaphragm. The diaphragm 1204 supports a substantially planar, ultrasonic transducer element 1210, which is adhesively bonded to an interior surface 1204b of the diaphragm 1204.

A ground and shielding electrode 1205 may be disposed on an interior surface 1204b of the diaphragm 1204, and may substantially cover this surface 1204b.

The transducer element 1210 may comprise a thin film of piezoelectric material including, without limitation, PVDF or PZT. A working electrode 1211 is disposed on an interior facing surface 1210b of the transducer element 1210, and may substantially cover this surface 1210b. The diaphragm facing surface 1210a of the transducer element 1210 is adhesively bonded to the interior surface 1204b of the diaphragm 1204. The bonding of the transducer element 1210 to the supporting diaphragm 1204 ensures that the transducer element 1210 is mechanically stressed in a manner which causes it to generate a voltage in response to an acoustic input applied thereto. An epoxy or other suitable adhesive bonding material, may be used for adhesively bonding the transducer element 1210 to the diaphragm 1204.

The housing wall section 1202 and the diaphragm 1204 are typically made from the same material used for making the housing 1201 of the electronic device, as the housing wall section 1202 is usually formed unitary with the housing 1201 of the electronic device. Such materials include, without limitation, electrically insulative materials, such as plastic, which can be formed using any conventional plastic forming method, such as plastic injection molding. It is contemplated that the housing wall section 1202 (and diaphragm 1204) of the EUMR 1200 may also be formed as a separate, discrete unit and combined with the rest of the housing 1201.

In operation, an ultrasonic wave propagating as shown by arrows, may propagate into the acoustic aperture 1203 causing the diaphragm 1204 to vibrate, the vibrations being detected by the transducer element 1210. Note that if the depth d is larger than one half of the wavelength λ, the diaphragm vibration becomes smaller due to cancellation of the acoustic signal at the top-most and bottom-most regions. For d<λ/2, λ=4 mm at 80 KHz operating frequency.

The EUMR shown in FIGS. 35A-B may be embodied as a separate, discrete, modular, ultrasonic micro-receiver (MUMR) 1200′ as shown in FIGS. 35C-D. The UMR 1200′ comprises a housing 1202′ having a transducer element 1210 is attached to a portion of a side wall 1204 which operates as a supporting diaphragm. The size and shape of the UMR's housing comports with the size and shape of corresponding slot or aperture A, defined, for example, by side walls A1 and A2, and bottom wall A3, formed in the exterior surface of a housing H an electronic device, which will receive the MUMR 1200′. The housing 1202′ of the MUMR 1200′ and the aperture A of the device housing H define an acoustic aperture 1203′ of a width w and a depth d (where w>d), which is capable of receiving of an acoustic signal S propagating along the exterior surface of the device. The device housing aperture A has depth G (where G>d), such that the top of the MUMR 1200′ is substantially flush with the exterior surface of the device housing H. The MUMR 1200′ may be mechanically secured within the aperture A, and electrically coupled via electrodes 1220 that pass through the bottom of the housing 1202′, to appropriate electronic circuitry (not shown) to provide an electrical output signal indicative of the incident acoustic waveform. The housing 1200′ of the MUMR 1200′ is preferably formed of the same material as that of the housing H of the electronic device including, without limitation, plastic or metal.

As shown in FIG. 35D, the transducer element 1210′ is adhesively bonded to the interior surface 1204b′ of side wall 1204′, which may be a discrete substrate, such as a printed circuit board (PCB), for example. The transducer element 1210′ may comprise a thin, film of piezoelectric material including, without limitation, PZT. Electrical connections 1221, such as wirebonds, operate to connect the transducer element 1210′ with corresponding conductive surfaces 1222 on the substrate 1204′ for providing signal connections.

FIGS. 35E-F collectively show an alternate embodiment of a MUMR, denoted by numeral 1200″. The MUMR 1200″ is similar to the MUMR of FIGS. 35C-D, except that the acoustic aperture 1203″ is formed in the top wall of the MUMR's housing 1202″, and the transducer element 1210″, comprises an outwardly curved or semi-cylindrical design. The outwardly curved transducer element 1210″ is disposed beneath the acoustic aperture 1203″. The transducer element 1210″ is adhesively bonded to an outwardly curved supporting member 1204″, which is mounted to a support plate 1230. The supporting member 1204″ may be made, for example, of a polyester material. The ends of the supporting member 1204″ are captured in slots defined in the support plate 1230″. Contact pins 1231″ capture the transducer element's electrode connections 1232″, and pass through the bottom of the housing 1202″ to connect with main circuitry (not shown). As with the MUMR of FIGS. 35C-D, the MUMR 1200″ is insertable into an aperture of a housing of an electronic device.

FIG. 36 shows still another embodiment of the EUMR of the present invention, denoted by numeral 1300. The EUMR 1300 is similar to the embodiment of FIGS. 35A-B, except that the side wall 1304 of the acoustic aperture 1303 of the EUMR 1300 comprises a metal plate, which operates as a supporting diaphragm. The metal plate 1304 may be made, for example of aluminum or stainless steel, and may have a thickness of about 0.1 mm. The metal plate may be affixed conventionally to the housing wall section 1302 using an adhesive or like means. The ultrasonic transducer element 1310 may comprise a thin film of piezoelectric material including, without limitation, PZT. A PZT-based transducer element 1310 typically has a thickness of about 0.1 mm to 0.2 mm. An epoxy or other suitable adhesive bonding material, may be used for adhesively bonding the transducer element 1310 to the metal plate diaphragm 1304. For an 80 KHz signal, the width w of the acoustic aperture 1303 may about 0.1 mm to 0.4 mm, the wall thickness t (the combined thickness of the diaphragm 1304 and transducer element 1310) may be about 0.2 mm, and the depth d of the acoustic aperture 1303 may be about 2 mm.

The EUMR of FIGS. 35A-B will now be described in greater detail with reference to FIG. 37, where the EUMR is denoted by numeral 1400. Note the EUMR 1400 of FIG. 37 is similar to the EUMR of FIG. 36, except that the EUMR 1400 has a diaphragm 1404, which forms a side wall of the acoustic aperture 1403, comprises a non-metal such as plastic and may have a thickness of about 0.4 mm. The ultrasonic transducer element 1410 may comprise a thin film of piezoelectric material including, without limitation, PVDF. A PVDF-based transducer element 1410 typically has a thickness of about 110 μm. An epoxy or other suitable adhesive bonding material, may be used for adhesively bonding the transducer element 1410 to the diaphragm 1404. For an 80 KHz signal, the width w of the acoustic aperture may about 0.1 mm to 0.4 mm, the wall thickness t (the combined thickness of the diaphragm and transducer element) may be about 0.2 mm, and the depth d of the acoustic aperture 1403 may be about 2 mm. Note that if a different operating frequency is used, the depth d has to be varied inversely proportional to the frequency from viewpoint of signal cancellation due to different phase at top and bottom. Also, the wall thickness t has to be varied to be proportional to the frequency such that a higher frequency requires a thinner wall thickness t. This is because resonant frequency is proportional to t/d².

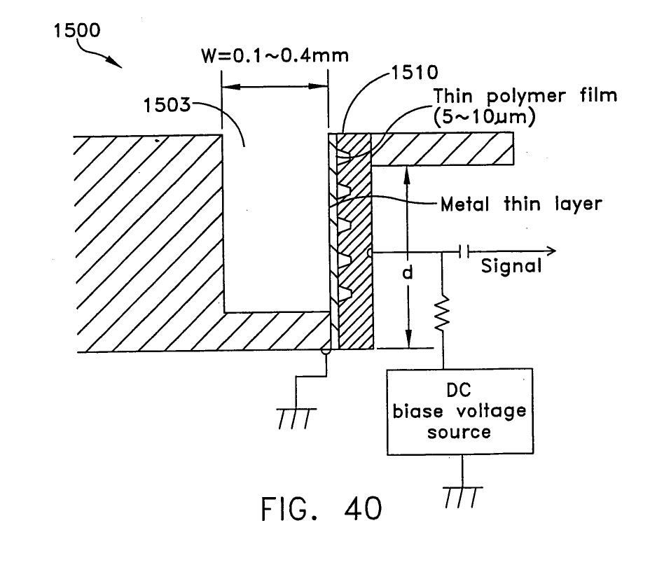

FIG. 40 shows still another embodiment of the EUMR of the present invention, denoted by numeral 1500. The EUMR 1500 is similar to the embodiments of FIGS. 35A-B, 36 and 37, except that the EUMR 1500 has an acoustic aperture side wall formed by an electrostatic transducer 1510 forming the sidewall of the acoustic aperture 1503. The electrostatic transducer 1510 comprises a thin metal film 1510a on which is disposed a thin polymer film 1510b having a thickness of between 5.0 μm-10.0 μm.

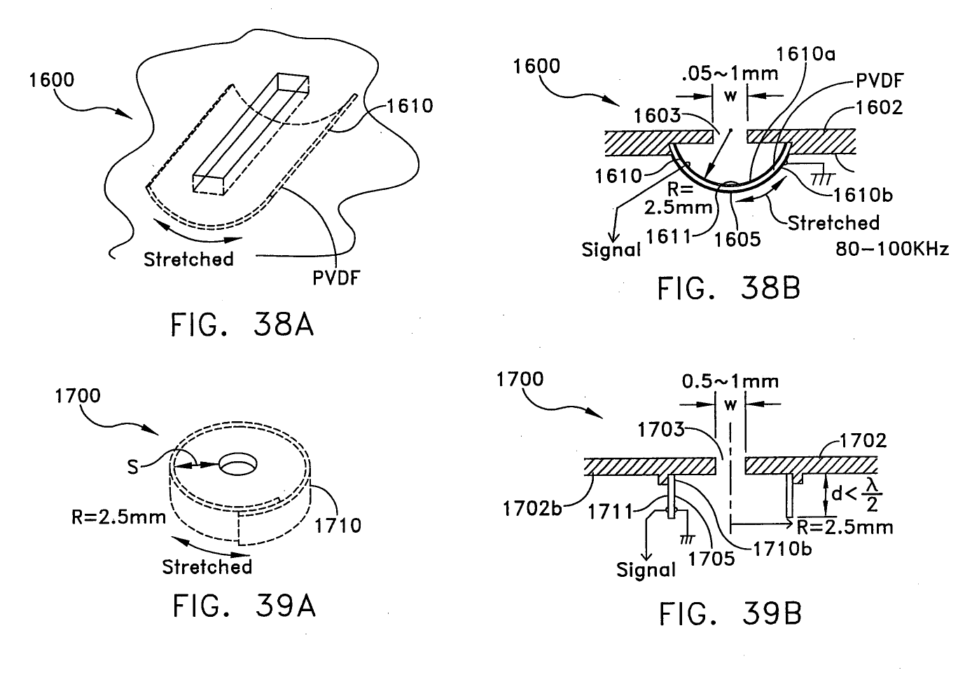

FIGS. 38A-B collectively show another alternative embodiment of the EUMR of the present invention, denoted by numeral 1600. The EUMR 1600 differs from the previous embodiments of the EUMR, as it comprises a transducer element 1610, formed as a downwardly curved cylindrical section, disposed below a centrally located rectangular acoustic aperture 1603. The transducer element 1610 may comprise a thin film of piezoelectric material including, without limitation, PVDF, which has been transversely stretched. A PVDF-based transducer element 1610 typically has a thickness of about 28 μm. A ground and shielding electrode 1605 may be disposed on an interior surface 1610b of the transducer element 1610, and a working electrode 1611 may be disposed on an exterior surface 1010a of the transducer element 1610. The electrodes may substantially cover the transducer surfaces 1610a, 1610b.

The lateral ends of the transducer element 1610 are affixed or clamped to the interior surface 1602b of the housing wall section 1602 in a manner that maintains the transducer element 1610 at a substantially constant distance radius R from the acoustic aperture 1603. This method of mounting ensures that the transducer element 1610 is mechanically stressed in a manner which causes it to generate a voltage in response to an acoustic input applied thereto. For operation in the range of 80-100 KHz, the radius R can be 2.5 mm. The radius R is inversely proportional to the frequency such that a higher frequency requires a smaller radius.

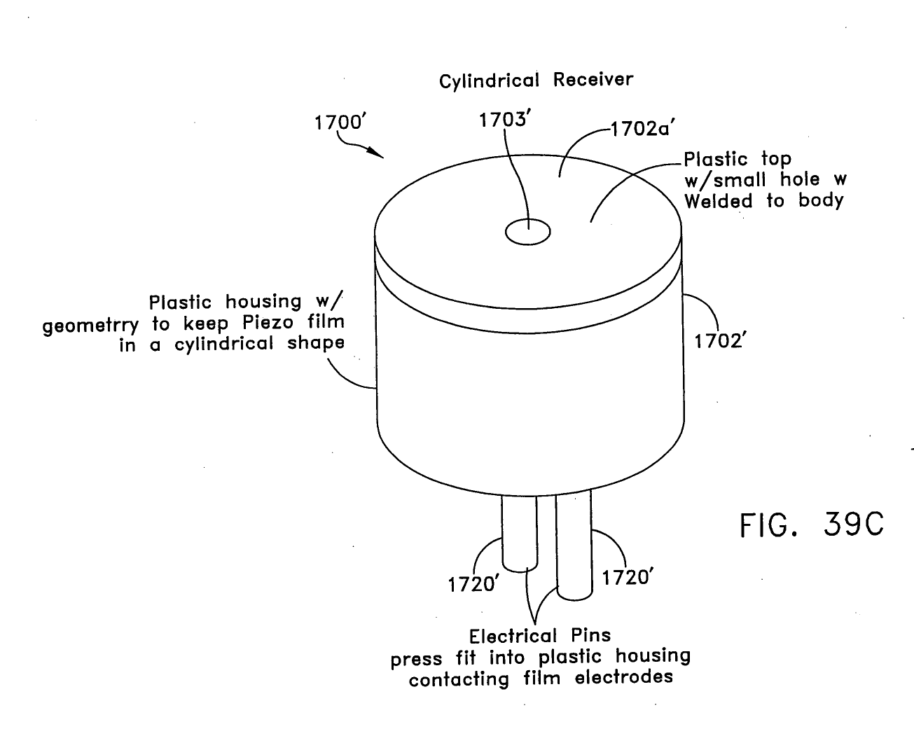

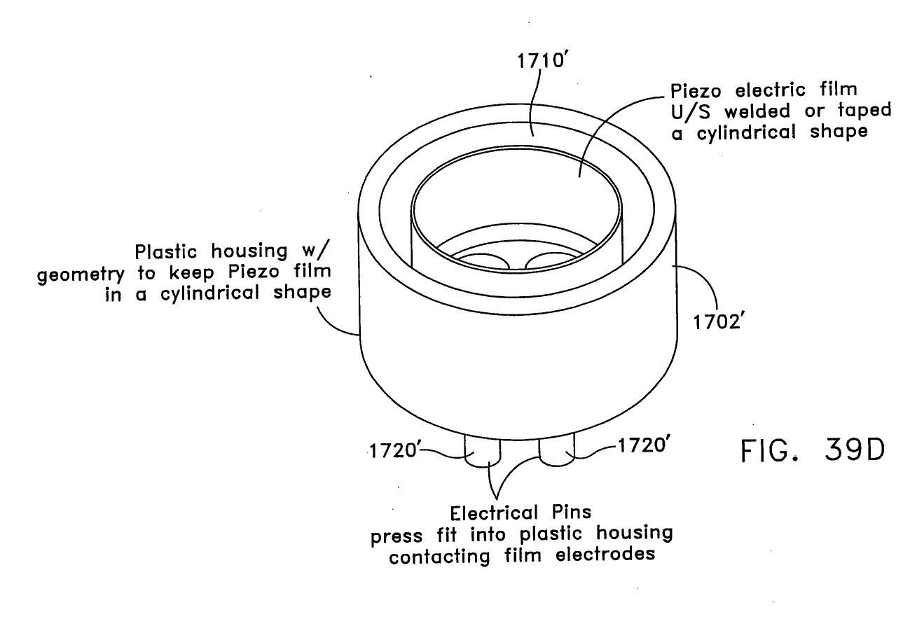

FIGS. 39A-B collectively show a further alternative embodiment of the EUMR of the present invention, denoted by numeral 1700. The EUMR 1700 differs from the previous embodiments of the EUMR by virtue of a transducer element 1710, formed as a cylinder attached to the interior surface 1702b of the housing wall section 1702, below a centrally located, circular acoustic aperture 1703 having a diameter w. The transducer element 1710, the ends of which overlap one another, maybe maintained in a cylindrical shape by ultrasonic welding, tape, or other adhesive and/or securing means. A working electrode 1711 may be disposed on an exterior cylindrical surface 1710a of the transducer element 1710 and a shielding and ground electrode 1705 may be disposed on the cylindrical interior surface 1710b of the transducer element 1710. The transducer element 1710 has a width d less than λ/2, which causes it to generate an electrical signal indicative of an impinging acoustic ultrasonic wave a propagating along the exterior surface 1702a of the housing wall section 1702. For a 80 KHz frequency of operation, the transducer element 1710 may have a radius R=2.5 mm as measured from the center of the acoustic aperture 1703 and the acoustic aperture 1703 may have a diameter w between 0.5 mm and 1.0 mm.

The EUMR shown in FIGS. 39A-B may be embodied as a separate, discrete ultrasonic micro-receiver (UMR) 1700′ as shown in FIGS. 39C and 39D. The UMR 1700′ comprises the earlier described transducer element 1710′ disposed in a cylindrical housing 1702′ made, for example, of a plastic material. The open end of the housing 1702′ is sealed with a cover 1702a′, which defines a circular acoustic aperture 1703′. The closed end of the housing 1702′ includes electrical pins 1720 which may be pressed fitted into the closed end of the housing 1702′. The pins 1720 contact the electrodes 1705′, 1711′ disposed on the surfaces of the transducer element 1710 contained in the housing 1702′. The UMR 1700′ may be integrated into a housing of an electronic device by inserting it into a correspondingly shaped aperture formed the electronic devices' housing.

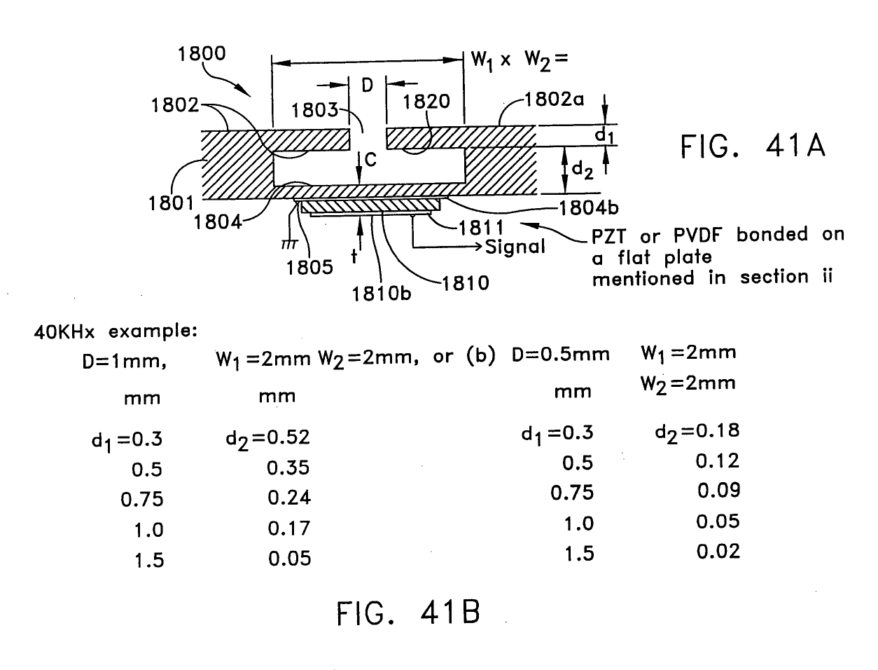

FIG. 41 A shows a further embodiment of the EUMR of the present invention, denoted by numeral 1800. The EUMR 1800 comprises a mono-morph structure formed by a housing wall section 1802 of a housing 1801 of an electronic device. The housing wall section 1802 defines a cavity C, the bottom wall 1804 of which defines a supporting diaphragm and the upper wall 1820 of which defines an acoustic aperture 1803 having a diameter d (0.5 mm to about 1.0 mm) that receives an ultrasound signal propagating along the exterior surface 1802a of the housing wall section 1802. The interior surface 1804b of the diaphragm 1804 has shielding and ground electrode 1805. A transducer element 1810 is adhesively bonded to the interior surface 1804b of the diaphragm 1804. A working electrode 1811 is formed on the interior surface 1810b of the transducer element 1810.

As shown in FIG. 41A, the cavity C has area defined by width w₁×width w₂. The upper wall 1820 of the cavity C has a thickness that defines a distance d1 and the cavity C has depth d2. The overall thickness of the bottom wall 1804 and transducer element 1810 is represented as t. The wall thickness, hole size D, and depth of the back space operate to provide impedance matching conditions.

The transducer element 1810 may comprise a thin film of piezoelectric material including, without limitation, PZT or PVDF. The transducer element 1810 typically has a thickness of about 0.1 to 0.5 mm. The bonding of the transducer element 1810 to the supporting diaphragm 1804 ensures that the transducer element 1810 is mechanically stressed in a manner which causes it to generate a voltage in response to an acoustic input applied thereto. An epoxy or other suitable adhesive bonding material, may be used for adhesively bonding the transducer element 1810 to the diaphragm 1804.

The housing wall section 1802 and cavity C are typically made from the same material used for making the housing 1801 of the electronic device, as the housing wall section 1802 is usually formed unitary with the housing 1801 of the electronic device. Such materials include, without limitation, electrically insulative materials, such as plastic, which can be formed using any conventional plastic forming method, such as plastic injection molding. It is contemplated that the housing wall section 1802 (and cavity C) of the EUMR 1800 may also be formed as a separate, discrete unit and combined with the rest of the housing 1801.

The table shown in FIG. 41B includes data for the EUMR 1800 embodied in FIG. 41A operating at 40 KHz frequency. Note that d1 and d2 are inversely proportional to the frequency of the device. Note further that additional geometries are contemplated in addition to square or rectangular structures, including for example a circular or cylindrical cavity C.

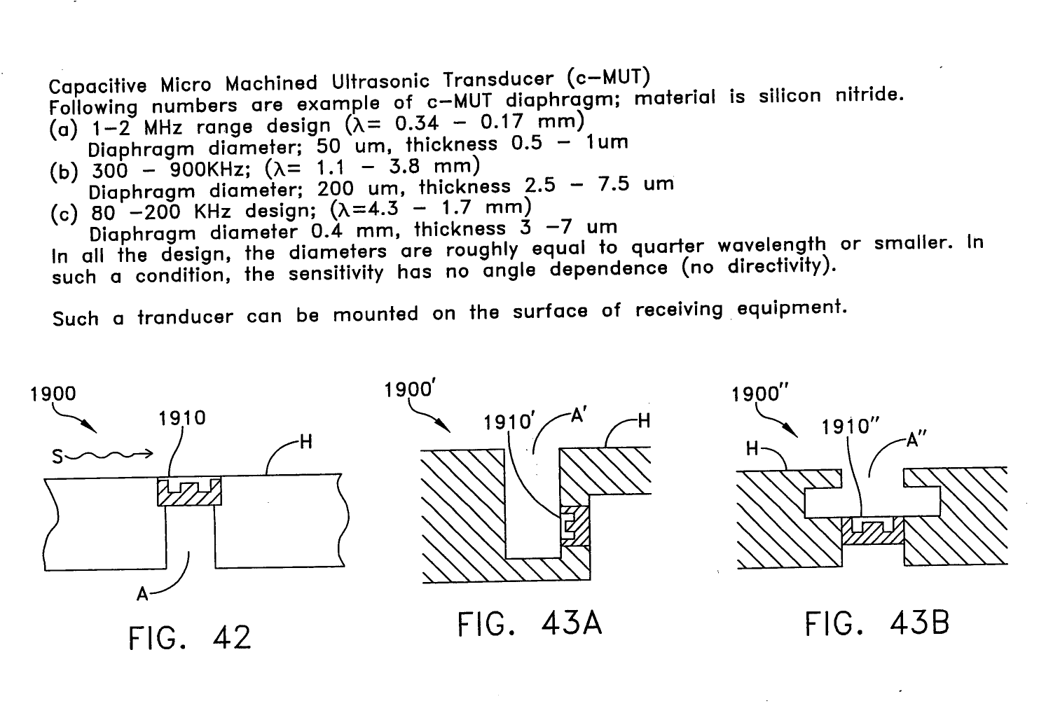

FIGS. 42, 43A, and 43B shown alternate embodiments of the MUMR of the present invention, which comprise a capacitive micro-machined ultrasonic transducer (CMUT) structure 1900, 1900′, 1900″ insertable into an aperture A, A′, A″ on an exterior surface of a housing H of an electronic device for receiving acoustic signals propagating along the surface of the device. The CMUT may include a circular diaphragm 1910, 1910′, 1910″, which may be made from micro-machinable material such as silicon nitride on silicon, for example. The CMUT may be bonded to the side walls of the aperture A, A′ as shown in FIGS. 42 and 43A, or to the bottom wall of aperture A″, as shown in FIGS. 43B. The impedance of the transducer is matched to air, obviating the need for a hole or slit.

Regarding the embodiment of FIG. 42, for a 1-2 MHz range CMUT MUMR where λ=0.34 mm-0.17 mm, the diameter of the diaphragm may be about 50 μm and the thickness about 0.5 μm-1.0 μm. For a 300-900 KHz range CMUT UMR where λ=1.1 mm-3.8 mm, the diameter of the diaphragm may be about 200 μm and the thickness about 2.5 μm-7.5 μm. For a 80-200 KHz range CMUT UMR where λ=4.3 mm-1.7 mm, the diameter of the diaphragm may be about 0.4 mm and the thickness about 3.0 μm-7.0 μm. The diameter of the diaphragm, in each embodiment may be roughly equal to a quarter wavelength λ or smaller. In such embodiments, the sensitivity has no angle dependence (no directivity).

Regarding the embodiments of FIGS. 43A-B, these structures may be used in place of the PZT or PVDF mono-morph structures discussed above. In first preferred embodiment, an 80-90 KHz CMUT MUMR may have a diaphragm size of about 24 μm in thickness and 2 mm in diameter. In second preferred embodiment, an 80-90 KHz CMUT UMR may have diaphragm size of about 12 μm in thickness and 1.4 mm in diameter. In third preferred embodiment, an 80-90 KHz CMUT UMR may have diaphragm size of about 6 μm in thickness and 1.0 mm in diameter. For the second and third embodiments identified above, such small diameters are comparable to one quarter wavelength in air so that the directivity of the transducer becomes very broad and the sensitivity of the acoustic wave impinging from any direction becomes essentially constant for different angles of incidence. Such transducer may then be mounted onto a flat surface so as to detect a surface propagating acoustic wave with minimal signal loss.

A third aspect of the present invention is a double-clamped ultrasonic transducer (DCUT) for hand-held, portable electronic devices of the type including, without limitation, cell phones, PDAs, notebook computers, micro-cassette recorders, and games. The DCUT may also be used for other types of electronic devices including, without limitation, keyboards used with personal computers. The DCUT may be integrated into the housing structure of the electronic device in manner which makes it virtually invisible from an external vantage point and makes it insusceptible to dust and dirt particles. The DCUT receives ultrasound signals propagating along a surface S of the electronic device or impinging normal to the surface of the transducer.

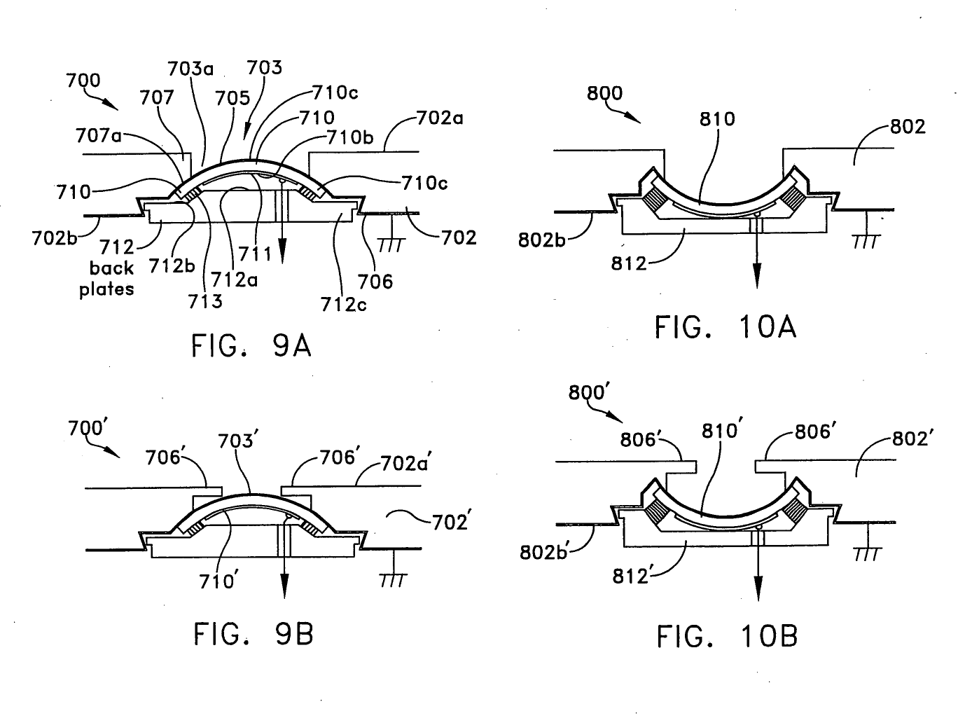

FIGS. 16-18 show an embodiment of a DCUT according to the present invention, denoted by numeral 2000. The DCUT 2000 comprises an outwardly curved transducer element 2010, which may comprise a film of piezoelectric material, such as PVDF, held in place by a protective front cover 2002 and a backplate 2012. The front cover 2002 and backplate 2012 are constructed to cooperate with one another to mechanically compress or clamp the ends of the transducer element 2010. The front cover 2002 includes curved clamping surfaces 2010b and the backplate 2012 includes corresponding curved clamping surfaces 2012b. The curvatures of the clamping surfaces 2010b, 2012b are substantially the same as the curvature of the transducer element 2010. The clamped end portions of the transducer element 2010 have about the same curvature as that of the main or central portion C of the transducer element 2010, which portion is not clamped or in any way attached to either the front cover 2002 or the backplate 2012. The main or central portion C of the transducer element 2010, defining the active area, and having a uniform, continuous curvature, is separated from the front cover 2002 and backplate 2012 by a front air gap 2020 and a back air gap 2030, respectively.

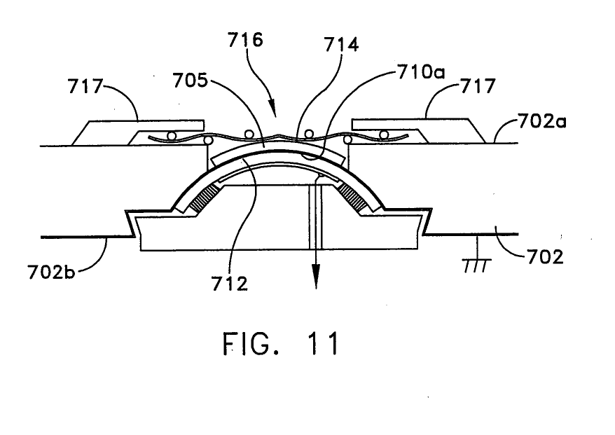

As best shown in FIG. 17, the backplate 2012 includes a generally planar base 2040 having a semi-cylindrical, outwardly curved member 2041 extending from a planar interior surface 2042. The outwardly curved member 2041 includes a recessed portion 2041a at its center, which defines curved lateral intermediate portions 2041b and curved transverse end portions 2041c. The curved transverse end portions 2041c form the curved clamping surfaces 2012b of the backplate 2012. The recessed portion 2041a at the center of the curved member 2041 and the curved lateral intermediate portions 2041b of the curved member 2041 enable the transducer element 2010 to move freely within the active area. Elongated transverse slots 2043 are defined in the interior surface 2042 of the base 2040, just beyond the curved transverse end portions 2041c of the curved member 2041. The slots 2043 receive corresponding snap engagement flanges 2053 extending from the cover 2002, and may in some embodiments, also receive the ends of the transducer element 2010.

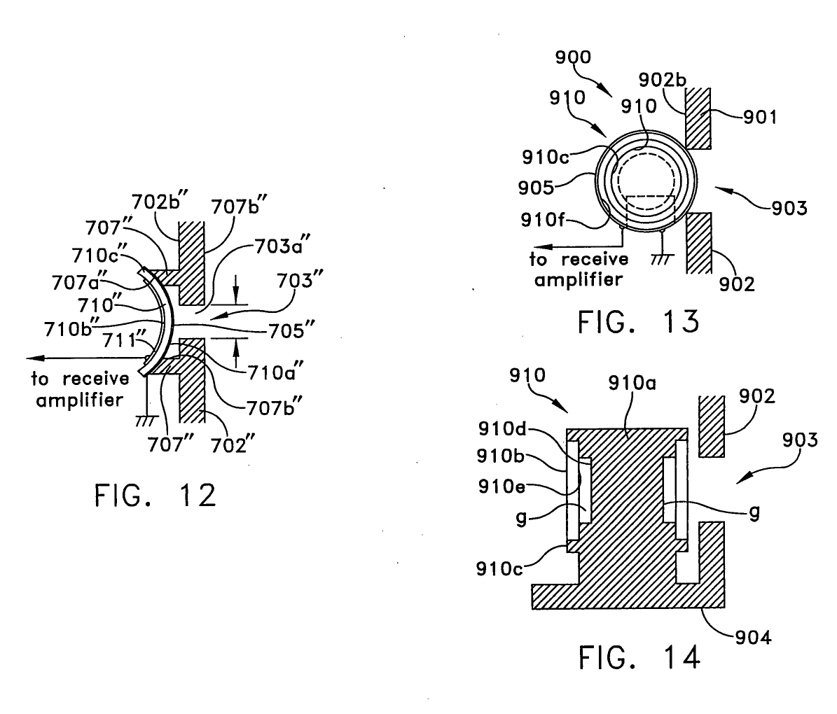

As best shown in FIG. 18, the front cover 2002 includes a generally planar top 2050 having a semi-cylindrical, inwardly curved member 2051 extending from a planar interior surface 2052 of the top 2050. The curved member 2051 includes a recessed protection grid 2051a at its center, which defines inwardly curved lateral intermediate portions 2051b and inwardly curved transverse end portions 2051c. The curved transverse end portions 2051c form the curved clamping surfaces 2010b of the front cover 2002. The recessed protection grid 2051a at the center of the curved member 2051 and the curved lateral intermediate portions 2051b of the curved member 2051 also enable the transducer element 2010 to move freely within the active area. The elongated snap engagement flanges 2053, mentioned-above, depend from the interior surface 2052 of the top 2050, just beyond the curved transverse end portions 2051c of the curved member 2051. The flanges 2053 may have inwardly extending hooks or barbs 2053a, which cooperate with recessed ledges 2043a defined at the bottom of the slots 2043 in the base 2040 of the backplate 2012 to secure the front cover 2002 and backplate 2012 to one another (FIG. 16). The interior surface 2052 of the top also include a pair of ribs 2052a disposed perpendicular to the flanges 2053, which cooperate with the backplate 2012.

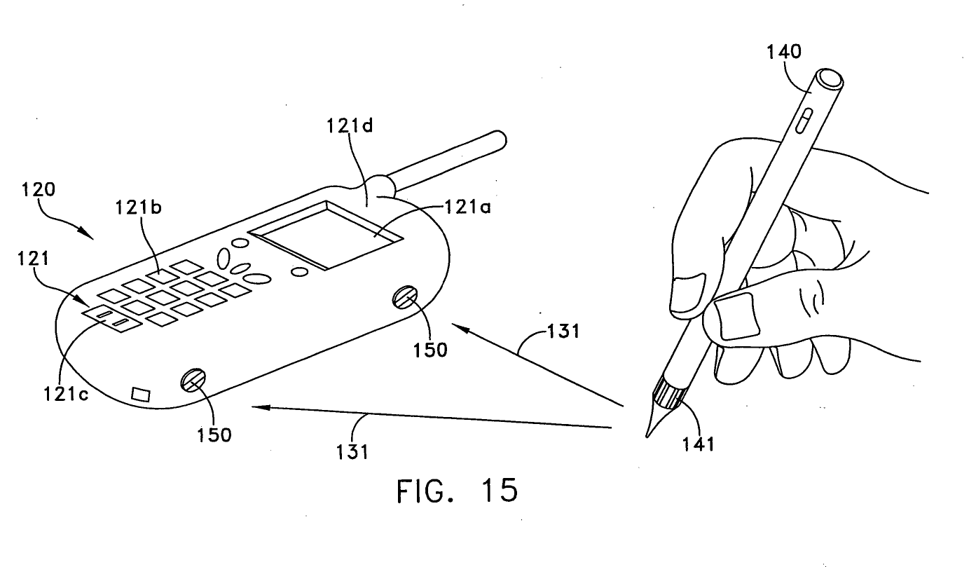

As shown in FIG. 19, the curved protection grid 2051a includes a series of elongated openings or slots 2060, preferably all of a uniform dimension, operate to protect the transducer element 2010 from damage due to external factors. In addition to providing protection from external sources, grid 2051a functions as an impedance matching element to the propagating acoustic signal. That is, the grid 2051a, is separated from the transducer element 2010 by the front air gap 2020, operates as an obstruction to an acoustic signal to cause a reflection therefrom, the reflected wave having a suitable phase creating a higher effective impedance value by loading to the transducer element 2010 and increasing the sensitivity of the device. Factors associated with the impedance matching function of the protection grid 2051a include the passage rate (ratio of open area to total active transducer element area d₂ (i.e. open area d₁+blocking area)), the distance between the protection grid 2051a and the transducer element 2010 (air gap 2020), and the dimension 2054 (or thickness) of the protection grid 2051a in the propagation direction. Typically, a passage rate of 40 to 60 percent is adequate and produces 50-80% improvement of sensitivity. It has been observed that for passage rates of 80%-90%, the efficient improvement decreases (<20%), and the mechanical strength is lessened.

FIG. 23 shows the calculated performance for various dimensions associated with the above parameters for an 80 KHz acoustic signal. For different operating frequencies, the dimension is changed inversely proportional to the frequency.

Referring collectively to FIGS. 16, 17 and 18, the portions of transducer element 2010 laying over the curved surface portions 2041b of the curved member 2041 are in contact engagement therewith. The curved transverse end portions 2041c, which define the clamping surfaces 2012a (FIG. 16) of the backplate 2012, urge the ends of the transducer element 2010 against the corresponding curved transverse end portions 2051c, which define the clamping surfaces 2002a (FIG. 16) of the front cover 2002, so as to clamp the ends of the transducer element 2010 therebetween. While the transducer element 2010 rests the curved surface portions 2041a of the backplate 2012, due to the slight tension originating therefrom, the transducer element 2010 is not clamped by portions 2041a. As mentioned earlier, the clamping surfaces 2012a, 2002a (FIG. 16) associated with the backplate 2012 and the front cover 2002 have the same curvatures. This enables both ease of manufacturing and enables the front cover 2002 and the backplate 2012 to maintain transducer curvature uniformity over the free or moving area (active area) of the transducer element 2010.

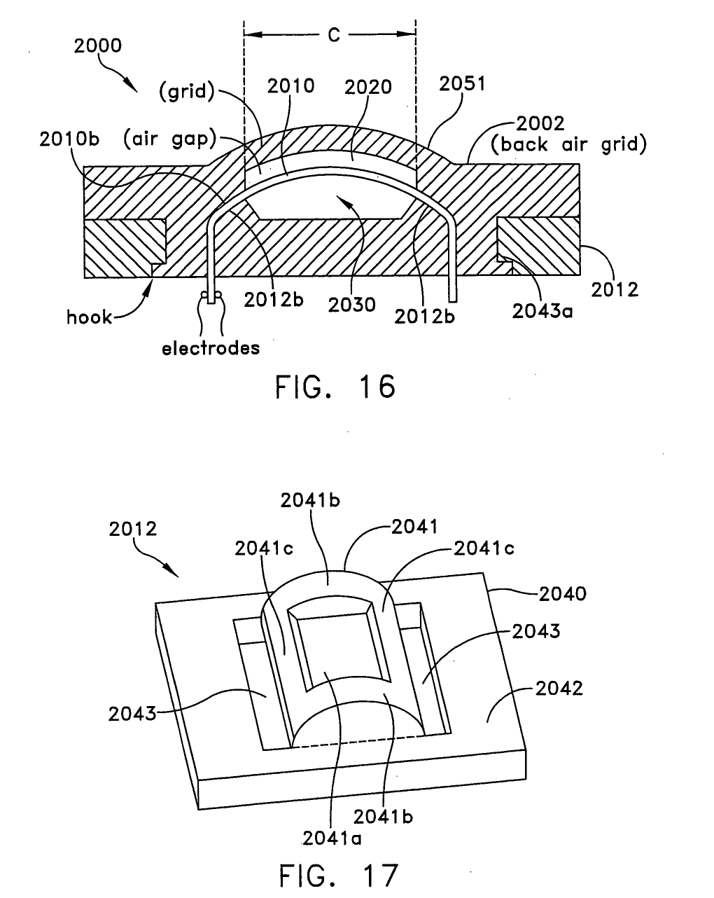

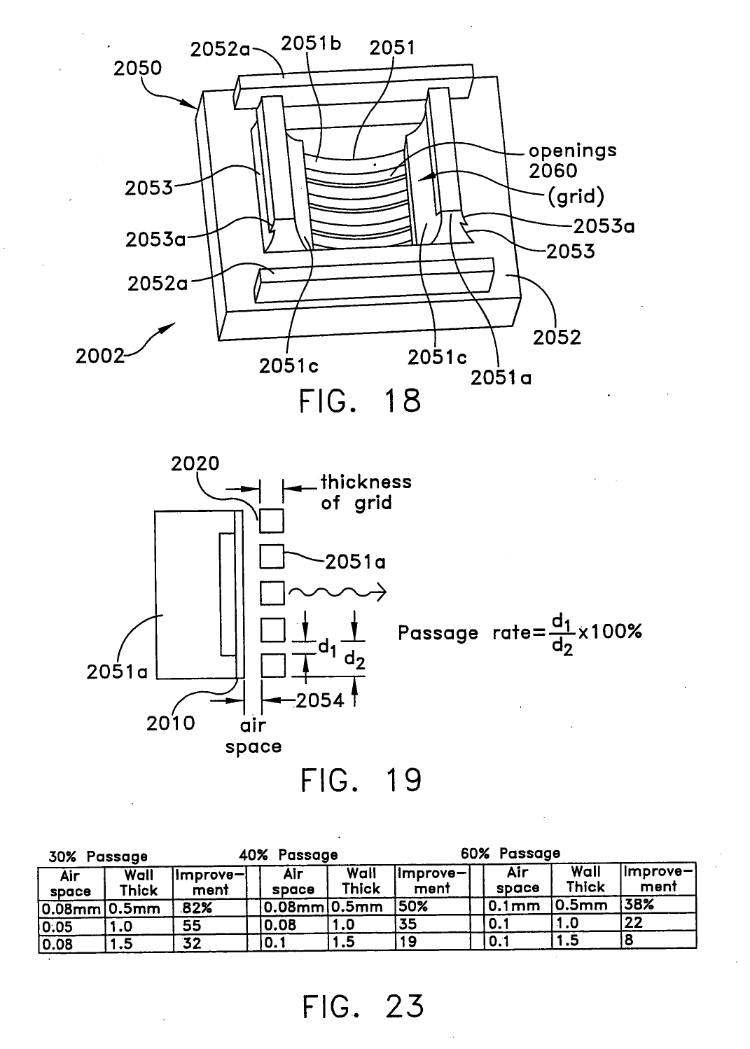

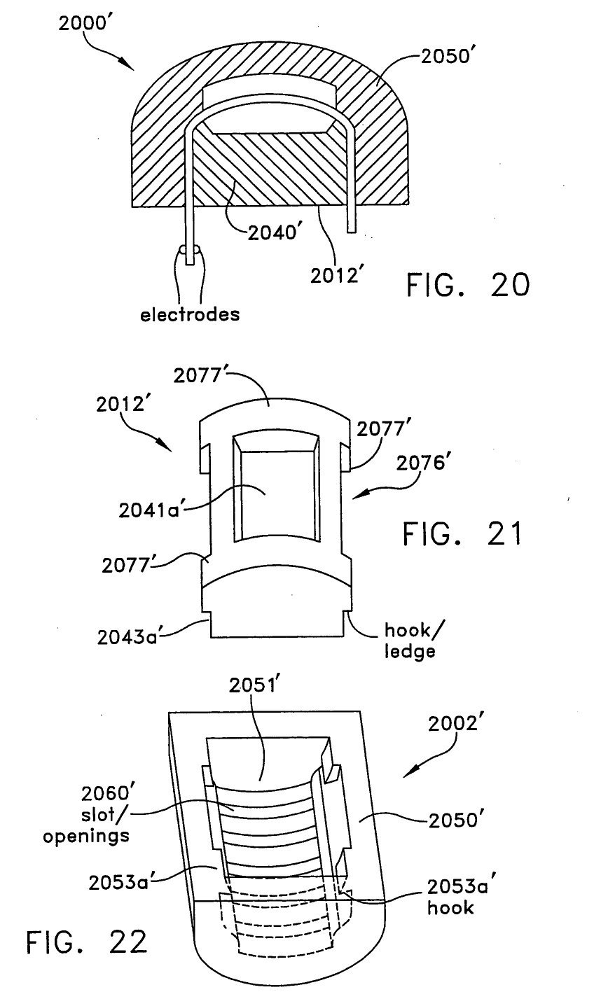

FIGS. 20-22 collectively show an alternative embodiment of a DCUT according to the present invention, denoted by numeral 2000′. The DCUT 2000′ is substantially similar to the DCUT 2000 depicted in FIG. 16, except that it has a more compact structure, with a shorter backplate base 2040′ and a shorter front cover top 2050. In addition, the backplate 2012′ DCUT includes end indent members 2076′ having indent portions 2077′, the bottom of the indents members 2076′ having the recessed ledges 2043a′ for snap engaging with hook member 2053a′ on the backside shortened front cover top 2050′.

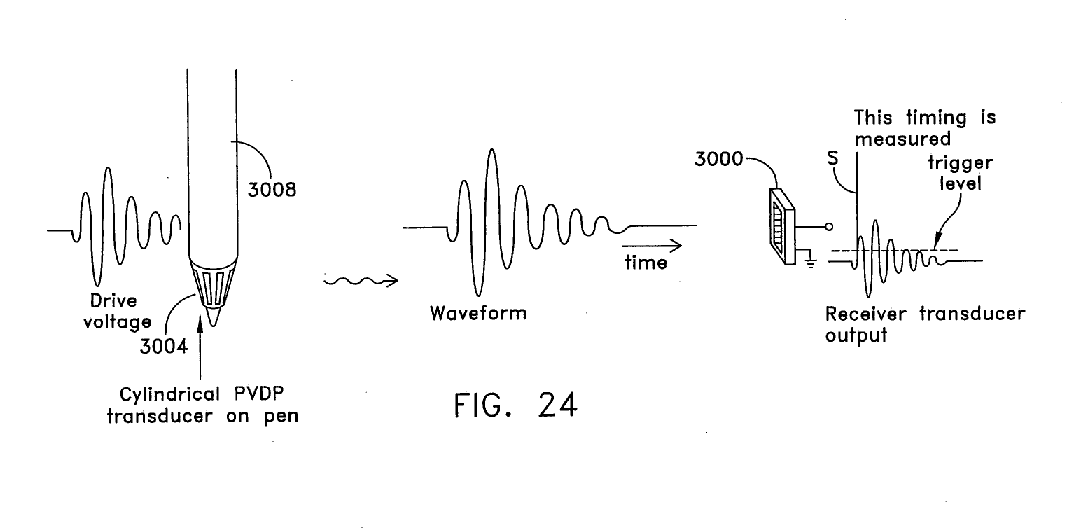

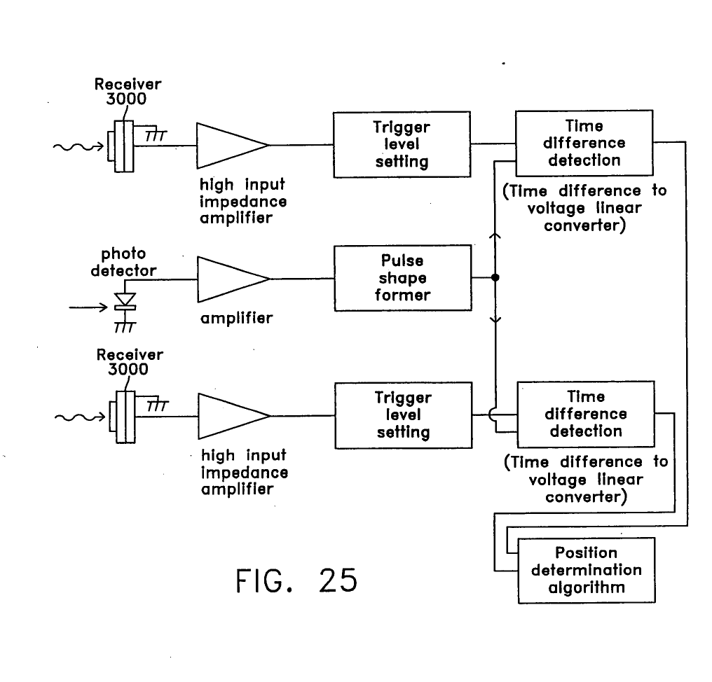

The aforementioned DCUT structures may be operated advantageously in a manner such that as a receiver they are responsive to a single, initial cycle of an acoustic waveform incident thereon for detecting the propagation delay time associated with a transmitter device 3004 mounted on a movable stylus 3008, as shown in FIG. 24. FIG. 24 schematically illustrates a drive voltage signal V imparted to ultrasound transmitter 3004 mounted on the tip of a movable stylus 3008 in response to movement of the stylus 3008 on a medium such as a piece of paper, for example. In response to the drive signal V, an acoustic waveform A is emitted from the ultrasound transmitter and propagates to the DCUT receiver 3000 fixedly positioned on/within a portable electronic device. The DCUT receiver output signal S output therefrom is processed by appropriate electronic circuitry such as, for example, that shown in FIG. 25, providing timing measurements and trigger level detection of the output signal to determine the relative position of the stylus 3008. Note that FIG. 25 illustrates an embodiment having two DCUT receivers 3000 processing acoustic information similar to the system illustrated in FIG. 14, with an optical signal from the stylus 3008 indicative of an initiation, termination or modification of acoustic data thereby operating as a controlling mechanism. Note also that, although it is preferred that the DCUT is employed as a receiver mounted on fixed equipment, it is also contemplated that the DCUT may also be used as a transmitter. The directivity of such a device, however, is not spread about a full 360 degrees (in the horizontal plane), but rather confined to about 50-60 degrees.

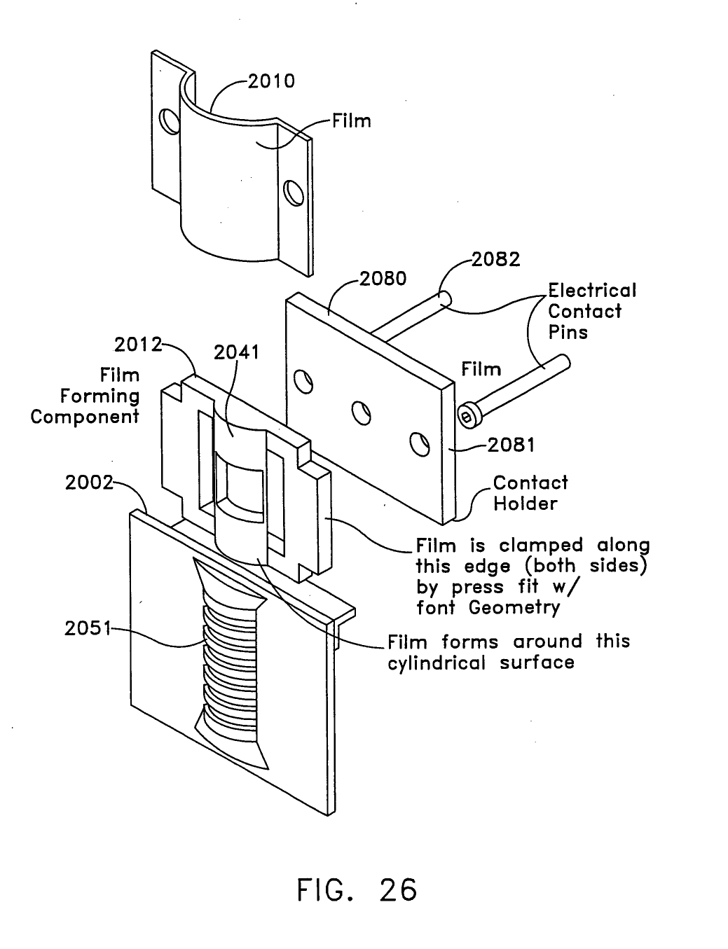

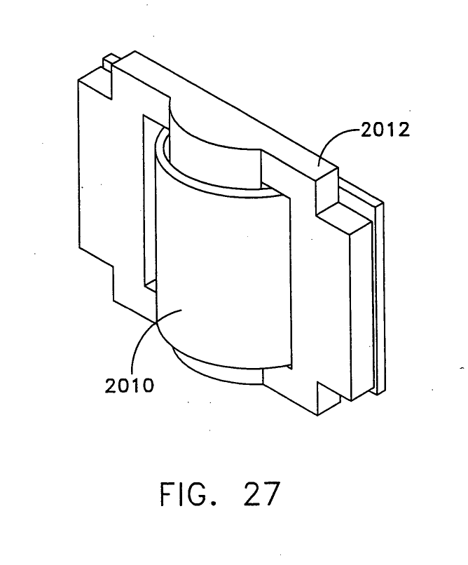

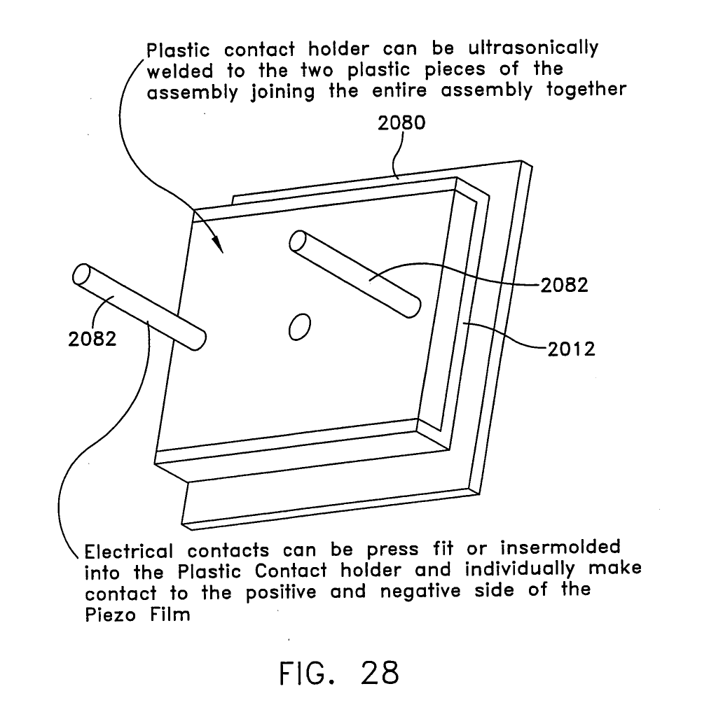

FIGS. 26-28 show the components of the DCUT 2000 depicted in FIGS. 16-18. FIG. 26 shows an exploded view of the DCUT 2000 including the front cover 2002 with the semi-cylindrical member 2051 including the protection grid 2051a, the backplate 2012 having the semi-cylindrical, curved member 2041 complementarily shaped with the semi-cylindrical member 2050, and the transducer element 2010. Also shown, is a contact holder 2080 comprising a generally planar member, may be coupled to the exterior surface 2012 of the backplate 2012, the contact holder 2080 having through holes 2081 for receiving electrical contact pins 2082 to provide electrical communication with an electronic device. FIG. 27 shows an assembled view of some of the components shown in FIG. 26, while FIG. 28 illustrates a rear perspective view thereof. The contact holder 2080, front cover 2002, and backplate 2012 can be formed of a non-metal material including, without limitation, rubber or plastic, and welded together using an ultrasonic welding method or the like to join the entire assembly. The electrical contact pins 2082 can be press fit or molded into the plastic contact holder 2080 and each coupled to a corresponding one of the working and shielding/ground electrodes (not shown) disposed on opposite surfaces of the transducer element 2012.

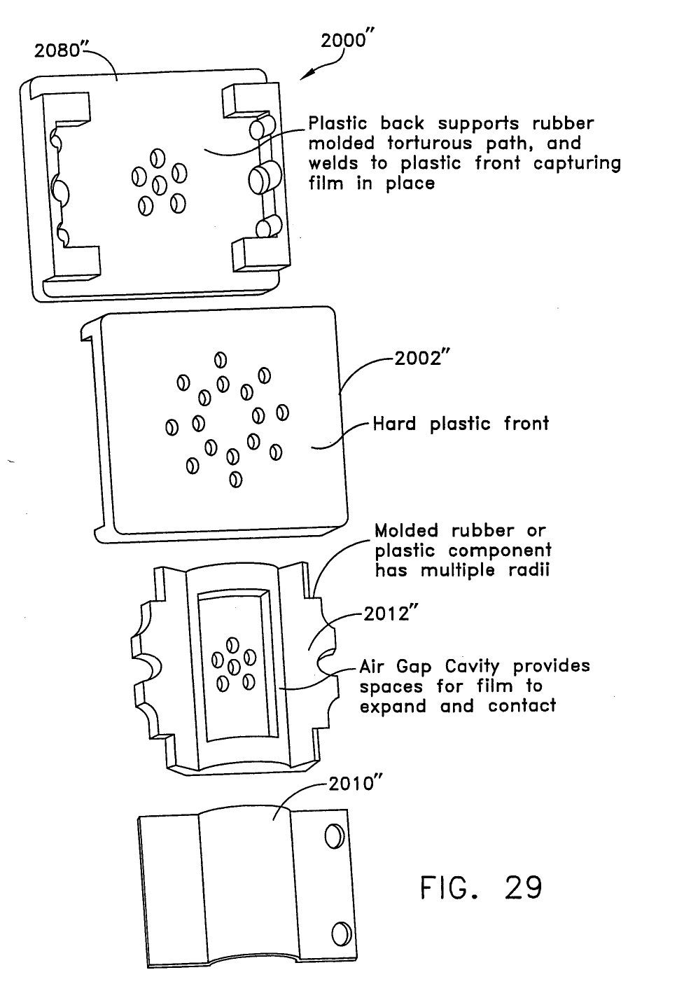

FIG. 29 shows an exploded view of a DCUT 2000″ according to still another embodiment of the present invention. The DCUT 2000″ comprises a front cover 2002″, a backplate 2012″, a transducer element 2010, and a contact holder 2080″.

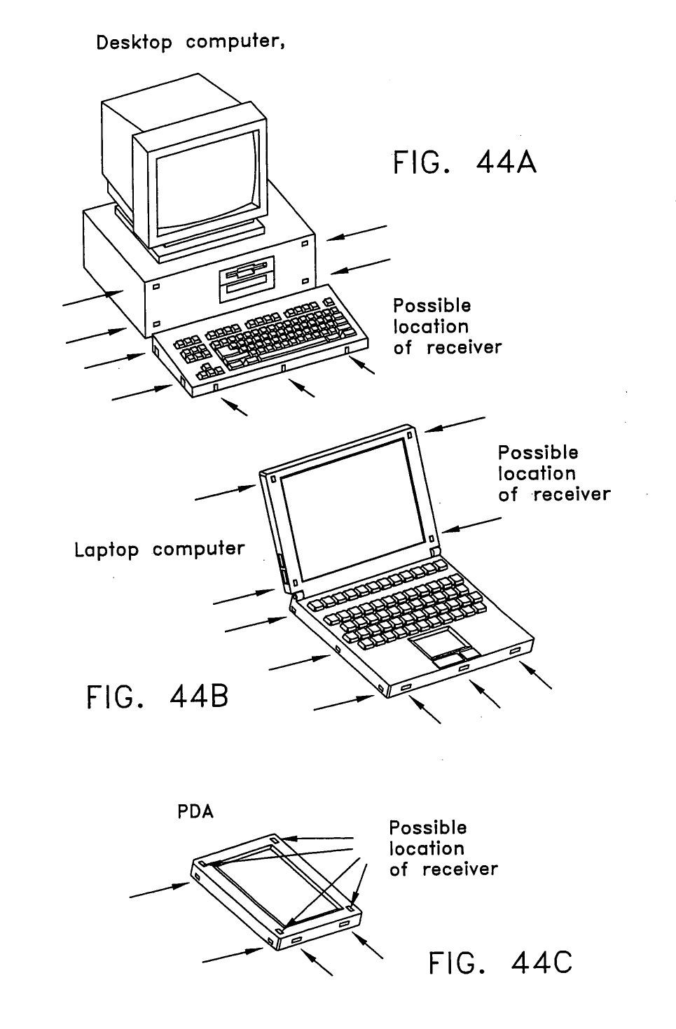

FIGS. 44A-C show a few exemplary computer applications where the ultrasonic transducers structures described herein can be utilized. FIG. 44A shows a desktop computer application of the ultrasonic transducer of the present invention illustrating a few exemplary locations for the transducers. FIG. 44B show a laptop computer application of the ultrasonic transducer of the present invention illustrating a few exemplary locations for the transducers. FIG. 44C shows PDA application of the ultrasonic transducer of the present invention illustrating a few exemplary housing locations for the transducers.

The foregoing inventions have been illustrated with embodiments including variously mounted transducer structures having acoustic apertures formed therein for receiving and detecting impinging ultrasound signals. Embodiments have exemplified the concept of an acoustic aperture and cavity wherein the depth of the cavity is controlled such that the dimensions of the cavity control a resonance frequency and hence increased sensitivity of the device. Various configurations, geometries and materials and dimensions have been illustrated. While the foregoing inventions have been described with reference to the above embodiments, various modifications and changes can be made without departing from the spirit of the invention.

1. An ultrasound receiver for an electronic device, the receiver comprising:
a housing;
an ultrasonic transducer element disposed in the housing, the transducer element responsive to an impinging acoustic signal and producing an output electrical signal representing an acoustic waveform defined by the impinging acoustic signal; and
electronic circuitry for processing the output signal;
wherein the receiver is responsive to a cycle of the acoustic waveform defined by the impinging acoustic signal.

2. The receiver according to claim 1, wherein the impinging acoustic signal is transmitted from a remotely located ultrasound transmitter.

3. The receiver according to claim 2, wherein the electronic circuitry includes:
timing measuring circuitry for measuring the timing of the waveform represented by the output signal; and
trigger level detection circuitry for detecting the single cycle of the waveform represented by the output signal;
the timing measuring and trigger level detection circuitry determining a position of the transmitter.

4. The receiver according to claim 1, wherein the electronic circuitry includes timing measuring circuitry for measuring the timing of the waveform represented by the output signal.

5. The receiver according to claim 1, wherein the electronic circuitry includes trigger level detection circuitry for detecting the single cycle of the waveform represented by the output signal.

6. The receiver according to claim 1, wherein the housing has at least one surface which ensures mechanical stressing of the ultrasonic transducer element, in response to the impinging acoustic signal, in a manner which causes the transducer element to produce the output signal.

7. The receiver according to claim 6, wherein the at least one surface of the housing clamps opposing ends of the transducer element.

8. The receiver according to claim 7, wherein the transducer element is curved.

9. The receiver according to claim 8, where the housing includes a front cover having an acoustic aperture.

10. The receiver according to claim 9, wherein the transducer element is disposed below the acoustic aperture.

11. The receiver according to claim 10, wherein the acoustic aperture includes a grid disposed across the aperture.

12. The receiver according to claim 11, wherein the grid operates as an impedance matching element to enable an acoustic sensitivity of the receiver to be increased.

13. The receiver according to claim 10, wherein the grid operates to protect the transducer element from an external environment.

14. The receiver according to claim 10, wherein the acoustic aperture is formed in an outwardly curved member.

15. The receiver according to claim 14, wherein the transducer element curves toward the acoustic aperture.

16. The receiver according to claim 9, wherein the housing further includes a backplate, the opposing ends of the transducer element being clamped between the front cover and the backplate.

17. The receiver according to claim 16, further comprising a holder for holding electrical contact pins which provide electrical communication between the transducer element and the electronic circuitry.

18. The receiver according to claim 17, wherein the at least one surface of the housing includes clamping surfaces defined by the front cover and the backplate.

19. The receiver according to claim 18, wherein the clamping surfaces are complementarily curved.

20. The receiver according to claim 19, wherein curvature of the clamping surfaces are substantially identical to the curvature of the transducer element.

21. The receiver according to claim 20, wherein the clamping surfaces are formed by a first semi-cylindrical member defined by the front cover and a second semi-cylindrical member defined by the backplate.

22. The receiver according to claim 1, wherein the housing is a selected wall section of a housing of the electronic device.

23. The receiver according to claim 1, wherein the electronic device is portable.

24. The receiver according to claim 6, wherein the transducer element is bonded to the at least one surface of the housing.

25. The receiver according to claim 24, wherein the transducer element is curved.

26. The receiver according to claim 24, wherein the transducer element is substantially flat.

27. The receiver according to claim 24, wherein the at least one surface is a diaphragm capable of vibrating in response to the impinging acoustic signal.

28. The receiver according to claim 27, wherein the housing includes an exterior surface, the diaphragm being flush with the exterior surface of the housing.

29. The receiver according to claim 27, wherein the housing includes an exterior surface, the diaphragm being substantially flush with the exterior surface of the housing.

30. The receiver according to claim 27, wherein the housing includes exterior and interior surfaces, the diaphragm being recessed from one of the exterior and interior surfaces of the housing.

31. The receiver according to claim 27, wherein the housing includes an exterior surface, the diaphragm is orthogonal to the exterior surface of the housing.

32. The receiver according claim 27, wherein the housing includes transducer receiving cavity having a bottom wall, the diaphragm forming the bottom wall of the transducer receiving cavity.

33. The receiver according to claim 27, wherein the housing includes an acoustic aperture having a side wall, the diaphragm forming the side wall of the acoustic aperture.

34. The receiver according to claim 27, wherein the housing includes an acoustic aperture having a bottom wall, the diaphragm forming the bottom wall of the acoustic aperture.

35. The receiver according to claim 27, wherein the housing includes an acoustic aperture opening into a cavity having a bottom wall, the diaphragm forming the bottom wall of the cavity.

36. The receiver according to claim 27, wherein the transducer element is bonded to one of an exterior and interior surface of the diaphragm.

37. The receiver according to claim 1, wherein the transducer element comprises a film of piezoelectric material.

38. The receiver according to claim 37, wherein the piezoelectric material is selected from the group consisting of polyvinylidene fluoride and lead-zirconate-titanate.

39. The receiver according to claim 1, wherein the transducer element comprises an electrostatic transducer.

40. The receiver according to claim 1, wherein the transducer element is cylindrical.

41. An ultrasound transducer for an electronic device, the transducer comprising:
a housing; and
an ultrasonic transducer element associated with the housing, the transducer element capable of operating in at least one of a receiver mode and transmitter mode, in the receiver mode the transducer element producing an electrical signal in response to an impinging acoustic signal and in the transmitter mode the transducer element producing an acoustic signal in response to an electrical signal applied thereto;
wherein the housing has at least one surface which ensures mechanical stressing of the transducer element in a manner which causes the transducer element to produce the signals.

42. The transducer according to claim 41, wherein the at least one surface of the housing clamps opposing ends of the transducer element.

43. The transducer according to claim 42, wherein the transducer element is curved.

44. The transducer according to claim 43, where the housing includes a front cover having an acoustic aperture.

45. The transducer according to claim 44, wherein the transducer element is disposed below the acoustic aperture.

46. The transducer according to claim 45, wherein the acoustic aperture includes a grid disposed across the aperture.

47. The transducer according to claim 46, wherein the grid operates as an impedance matching element to enable an acoustic sensitivity of the transducer to be increased.

48. The transducer according to claim 45, wherein the grid operates to protect the transducer element from an external environment.

49. The transducer according to claim 45, wherein the acoustic aperture is formed in an outwardly curved member.

50. The transducer according to claim 49, wherein the transducer element curves toward the acoustic aperture.

51. The transducer according to claim 44, wherein the housing further includes a backplate, the opposing ends of the transducer element being clamped between the front cover and the backplate.

52. The transducer according to claim 51, further comprising a holder for holding electrical contact pins which provide electrical communication between the transducer element and the electronic circuitry.

53. The transducer according to claim 52, wherein the at least one surface of the housing includes clamping surfaces defined by the front cover and the backplate.

54. The transducer according to claim 53, wherein the clamping surfaces are complementarily curved.

55. The transducer according to claim 54, wherein curvature of the clamping surfaces are substantially identical to the curvature of the transducer element.

56. The transducer according to claim 55, wherein the clamping surfaces are formed by a first semi-cylindrical member defined by the front cover and a second semi-cylindrical member defined by the backplate.

57. The transducer according to claim 41, wherein the housing is a selected wall section of a housing of the electronic device.

58. The transducer according to claim 41, wherein the electronic device is portable.

59. The transducer according to claim 41, wherein the transducer element is bonded to the at least one surface of the housing.

60. The transducer according to claim 59, wherein the transducer element is curved.

61. The transducer according to claim 59, wherein the transducer element is substantially flat.

62. The transducer according to claim 59, wherein the at least one surface is a diaphragm capable of vibrating in response to the impinging acoustic signal.

63. The transducer according to claim 62, wherein the housing includes an exterior surface, the diaphragm being flush with the exterior surface of the housing.

64. The transducer according to claim 62, wherein the housing includes an exterior surface, the diaphragm being substantially flush with the exterior surface of the housing.

65. The transducer according to claim 62, wherein the housing includes exterior and interior surfaces, the diaphragm being recessed from one of the exterior and interior surfaces of the housing.

66. The transducer according to claim 62, wherein the housing includes an exterior surface, the diaphragm is orthogonal to the exterior surface of the housing.

67. The transducer according claim 62, wherein the housing includes transducer receiving cavity having a bottom wall, the diaphragm forming the bottom wall of the transducer receiving cavity.

68. The transducer according to claim 62, wherein the housing includes an acoustic aperture having a side wall, the diaphragm forming the side wall of the acoustic aperture.

69. The transducer according to claim 62, wherein the housing includes an acoustic aperture having a bottom wall, the diaphragm forming the bottom wall of the acoustic aperture.

70. The transducer according to claim 62, wherein the housing includes an acoustic aperture opening into a cavity having a bottom wall, the diaphragm forming the bottom wall of the cavity.

71. The transducer according to claim 62, wherein the transducer element is bonded to one of an exterior and interior surface of the diaphragm.

72. The transducer according to claim 41, wherein the transducer element comprises a film of piezoelectric material.

73. The transducer according to claim 72, wherein the piezoelectric material is selected from the group consisting of polyvinylidene fluoride and lead-zirconate-titanate.

74. The transducer according to claim 41, wherein the transducer element comprises an electrostatic transducer.

75. The transducer according to claim 41, wherein the transducer element is cylindrical.

76. The transducer according to claim 41, wherein the housing is separate from a housing of the electronic device.

77. The transducer according to claim 76, wherein the transducer is a modular unit insertable in an aperture of the housing of the electronic device.

78. The transducer according to claim 41, wherein the transducer element is curved.

79. The transducer according to claim 78, further comprising a backplate for clamping the transducer element to the housing.

80. The transducer according to claim 79, wherein the transducer element and backplate are disposed vertically relative to the housing.

81. The transducer according to claim 79, wherein the transducer element and backplate are disposed horizontally relative to the housing.

82. The transducer according to claim 81, wherein the housing includes an exterior surface having a recess formed therein which slopes toward the transducer element.

83. The receiver according to claim 1, wherein the transducer element is curved.

84. The receiver according to claim 83, further comprising a backplate for clamping the transducer element to the housing.

85. The receiver according to claim 84, wherein the transducer element and backplate are disposed vertically relative to the housing.

86. The receiver according to claim 84, wherein the transducer element and backplate are disposed horizontally relative to the housing.

87. The receiver according to claim 86, wherein the housing includes an exterior surface having a recess formed therein which slopes toward the transducer element.
